(19) United States
(12) Patent Application Publication
(10) Pub. No.: US 2024/0295064 A1
(43) Pub. Date: Sep. 5, 2024

(54) INTERFACE AND LAUNDRY TREATING APPARATUS HAVING THE SAME

(71) Applicant: LG ELECTRONICS INC., Seoul (KR)

(72) Inventor: Dami CHOE, Seoul (KR)

(73) Assignee: LG ELECTRONICS INC., Seoul (KR)

(21) Appl. No.: 18/575,388

(22) PCT Filed: Jun. 30, 2022
(86) PCT No.: PCT/KR2022/009432

§ 371 Date: Dec. 29, 2023

(30) Foreign Application Priority Data
Jun. 30, 2021 (KR) 10-2021-0085830
Jun. 30, 2021 (KR) 10-2021-0085831

(51) Int. Cl. D06F 34/32; A61L 2/07; A61L 2/24; D06F 25/00; D06F 34/18; D06F 34/30; D06F 39/08; D06F 39/40; D06F 58/20; D06F 103/08; D06F 103/62; D06F 105/40; D06F 105/52; D06F 105/58

(52) U.S. Cl. CPC D06F 34/32; A61L 2/07; A61L 2/24; D06F 25/00; D06F 34/18; D06F 34/30; D06F 39/088; D06F 39/40; D06F 58/203; A61L 2202/11; A61L 2202/122; A61L 2202/17; A61L 2202/26; D06F 2103/08; D06F 2103/62; D06F 2105/40; D06F 2105/52; D06F 2105/58

(57) ABSTRACT

A laundry treating apparatus including a cabinet, a drum provided inside the cabinet to accommodate laundry, a driver to provide power to rotate the drum, a steam part to supply steam to an inside of the drum, a water supply to supply water to the steam part, a manipulation portion to receive a command for selecting a course or option for performing one or more of rotation of the drum or a supply of steam to the inside of the drum, a controller to generate insufficiency information that steam is not capable of being generated, and a display portion to display the insufficiency information. The display portion can display the insufficiency information at any one of a booting time point at which power is supplied to the display portion, a selection time point of the course or option, an execution time point at which the course or option is executed, and a completion time point at which the course or option is completed, when the insufficiency information is generated.

### CROSS-REFERENCE TO RELATED APPLICATIONS

This application is a National Phase application under 35 U.S.C. 371 of International Application No. PCT/KR2022/009432, filed on Jun. 30, 2022, which claims the benefit of Korean Application No. 10-2021-0085830, filed on Jun. 30, 2021, and Korean Application No. 10-2021-0085831, filed on Jun. 30, 2021, the contents of which are incorporated by reference herein in their entirety.

### BACKGROUND OF THE INVENTION

### Technical Field

This application relates to an interface and a laundry treating apparatus having an interface.

### Background Art

An input/output device (e.g., an interface) for a user to input a control command to an electronic device is essential in the electronic device including a laundry treating apparatus capable of washing, drying, and washing and drying laundry (e.g., a common name of an object to be washed or an object to be dried).

The conventional interfaces were designed such that a display portion that displays a control command that a user may select, a search interface that allows the user to search for the control command displayed on the display portion, a selection portion that allows the user to select the control command displayed on the display portion, and input means that requests execution of the control command selected by the user are located in spaces separated from each other (KR 10-2014-0023986).

That is, in the conventional interface, a display (e.g., an LCD, an LED panel, and the like) on which the control command is displayed, a knob or a button that may search for the control command by allowing a control command executable by the corresponding electronic device to be displayed on the display, a button that inputs a control command to select the control command displayed on the display, a button that inputs a control command to request execution of the selected control command, and the like are located in spaces separated from each other. Such an arrangement was a factor determining a design of the interface (e.g., a design of the control panel) located on a front face of the electronic device such as the laundry treating apparatus.

In the conventional interface of the above structure, because the devices including the display had to be dispersedly arranged along a width direction of the electronic device or distributed along a height direction of the electronic device, a great space was required for installation of the interface.

The conventional laundry treating apparatus includes a steam part for supplying steam to a drum that accommodates laundry therein. The steam has an effect of sterilizing laundry, removing wrinkles from the laundry, and preventing the laundry from shrinking.

However, when water is not supplied to the steam part, the conventional laundry treating apparatus has no method of reliably indicating this.

When water is not supplied to the steam part, the conventional laundry treating apparatus has not reference as to when this needs to be indicated.

When water is not supplied to the steam part, the conventional laundry treating apparatus has a risk that the course and option using steam are interrupted, thereby delaying a cycle of processing laundry.

The conventional laundry treating apparatus does not separately provide an additional special course other than an existing course for washing laundry with water and detergent or drying with hot air.

In addition, the conventional laundry treating apparatus has a problem in that even if an additional course is developed due to the development of technology or installation of additional parts, a user is not capable of using the course because the user does not know or understand existence of the additional course itself.

There is a problem in that the conventional laundry treating apparatus does not provide an interface method for a user to select an existing course and an additionally developed course.

In particular, when there is conflict between the existing course and the additional course, there is a problem that is not capable of providing a solution on how to improve it.

### SUMMARY OF THE DISCLOSURE

### Technical Problem

An object of the present disclosure is to provide a laundry treating apparatus for recognizing a user when water is not supplied to a steam part for supplying steam.

An object of the present disclosure is to provide a laundry treating apparatus for blocking delay of a cycle for processing laundry even if water is not supplied to the steam part.

An object of the present disclosure is to provide a laundry treating apparatus for inducing an action of a user when a problem with water supply to a steam part is not repeatedly overcome.

An object of the present disclosure is to provide a laundry treating apparatus for immediately exposing an additionally developed course to a user and inducing the user to know the course.

An object of the present disclosure is to provide a laundry treating apparatus for changing a method of displaying an additionally developed course and an existing course.

An object of the present disclosure is to provide a control method in which an additionally developed course and an existing course do not collide with each other and are used interchangeably.

### Technical Solution

To achieve the object of the present disclosure, the present disclosure provides a laundry treating apparatus including a cabinet, a drum rotatably provided inside the cabinet and accommodating laundry, a driver connected or coupled to drum and providing power for rotating the drum, a steam part provided inside the cabinet and supplying steam to an inside of the drum, a supply connected to the steam part and supplying water to the steam part, a manipulation portion configured to receive a command for selecting an arbitrary course or option for performing one or more of rotation of the drum or supply of steam to the inside of the drum by operating the driver, a controller configured to generate insufficiency information that steam is not capable of being generated by detecting a state in which water supply for generating steam to the steam part is blocked or water supply is insufficient inside the steam part, and a display portion coupled to the cabinet and configured to display the insufficiency information to an outside.

The display portion may be controlled to display the insufficiency information at at least one of a booting time point at which power is supplied to the display portion, a selection time point of a course or an option of input of the manipulation portion, an execution time point at which the course or the option is executed, or a completion time point at which the course or the option is completed, when the insufficiency information is generated.

The laundry treating apparatus may further include a storage configured to store the insufficiency information, and when the insufficiency information is present in the storage at the booting time point, the display portion may display the insufficiency information at the booting time point.

The display portion may remove display of the insufficiency information when a reference time elapses from the booting time point.

The arbitrary course or option may further include a steam course or a steam option for necessarily driving the steam part, and when the steam course is selected through the manipulation portion, the display portion may display the insufficiency information at the selection time point.

The laundry treating apparatus may further include an executing portion configured to receive an execution command for executing the selected course or option, wherein, when the executing portion is input, the display portion may remove display of the insufficiency information and may display an execution state of the steam course or the steam option.

The steam course or the steam option may be completely executed without change even when the insufficiency information is displayed on the display portion.

When the insufficiency information is displayed on the display portion, the steam course may be stopped, and when display of the insufficiency information is removed from the display portion, the steam course may be executed again.

The manipulation portion may further include a selection portion configured to receive a command that a user confirms the insufficiency information, and the display portion may remove display of the insufficiency information upon receiving input of the selection portion.

The steam course may be stopped up to input of the executing portion or the selection portion.

The arbitrary course or option may further include a steam course or steam option for necessarily driving the steam part, and when the controller generates the insufficiency information during a process in which the steam course or the steam option is executed, the display portion may display the insufficiency information at the execution time point.

The display portion may display the insufficiency information at the execution time point, and may remove display of the insufficiency information when a reference time elapses.

The laundry treating apparatus may further include a storage configured to store the insufficiency information, and when the insufficiency information is maintained up to the completion time point, the controller may store the insufficiency information in the storage.

The controller may block power to one or more of the display portion and the driver after storing the insufficiency information in the storage.

When the steam part is capable of generating steam or water is supplied to the supply, the display portion may remove display of the insufficiency information.

### Advantageous Effects

The present disclosure may have an effect of recognizing a user when water is not supplied to a steam part for supplying steam.

The present disclosure may have an effect of preventing delay of a cycle for processing laundry even if water is not supplied to the steam part.

The present disclosure may have an effect of inducing an action of a user when a problem with water supply to a steam part is not repeatedly overcome.

The present disclosure may have an effect of immediately exposing an additionally developed course to a user and inducing the user to know the course.

The present disclosure may have an effect of changing a method of displaying an additionally developed course and an existing course.

The present disclosure may have an effect whereby an additionally developed course and an existing course do not collide with each other and are used interchangeably.

### BRIEF DESCRIPTION OF THE DRAWINGS

The present invention will become more fully understood from the detailed description given hereinbelow and the accompanying drawings which are given by way of illustration only, and thus are not limitative of the present invention.

FIGS. 1 and 2 show an example of an interface and a laundry treating apparatus including the interface.

FIG. 3 shows an internal structure of a laundry treating apparatus according to the present disclosure.

FIG. 4 shows a washing structure of a laundry treating apparatus according to the present disclosure.

FIG. 5 shows a steam supply structure of a laundry treating apparatus according to the present disclosure.

FIG. 6 shows as structure of a control panel.

FIG. 7 shows a structure of an encoder.

FIG. 8 shows a structure of the manipulation means P7 and the displaying interface P8.

FIG. 9 shows an embodiment in which the control panel P of a laundry treating apparatus according to the present disclosure is used.

(a)-(d) of FIG. 10 show examples of the control panel P during an operation of a laundry treating apparatus.

(a)-(d) of FIG. 11 show utilization of the displaying interface P8 and emphasizes the display p84 in the displaying interface P8.

(a)-(e) of FIG. 12 show an embodiment in which a course or an option is selected.

FIG. 13 shows a notification manner according to an operation process of a laundry treating apparatus according to the present disclosure.

FIG. 14 shows a control block diagram of a laundry treating apparatus according to the present disclosure.

(a)-(c) of FIG. 15 show an embodiment in which the insufficiency information is displayed.

(a) and (b) of FIG. 16 shows another embodiment of displaying the insufficiency information.

FIG. 17 shows another embodiment of displaying the insufficiency information.

FIG. 18 shows an embodiment of a control method of displaying the insufficiency information.

(a) and (b) of FIG. 19 show a special course and a method of selecting the special course in a laundry treating apparatus according to the present disclosure.

(a) and (b) FIG. 20 shows an additional embodiment of selecting a special course.

(a)-(d) of FIG. 21 show a control method to be selected and performed when the special course and the existing course are executed.

FIG. 22 shows an embodiment of a control method using both of a special course and an existing course by a laundry treating apparatus according to the present disclosure.

### DETAILED DESCRIPTION OF THE EMBODIMENTS

The present invention will now be described with reference to the accompanying drawings, wherein the same reference numerals have been used to identify the same or similar elements throughout the several views.

Hereinafter, a preferred embodiment of an interface and a laundry treating apparatus having the interface will be described in detail with reference to the accompanying drawings.

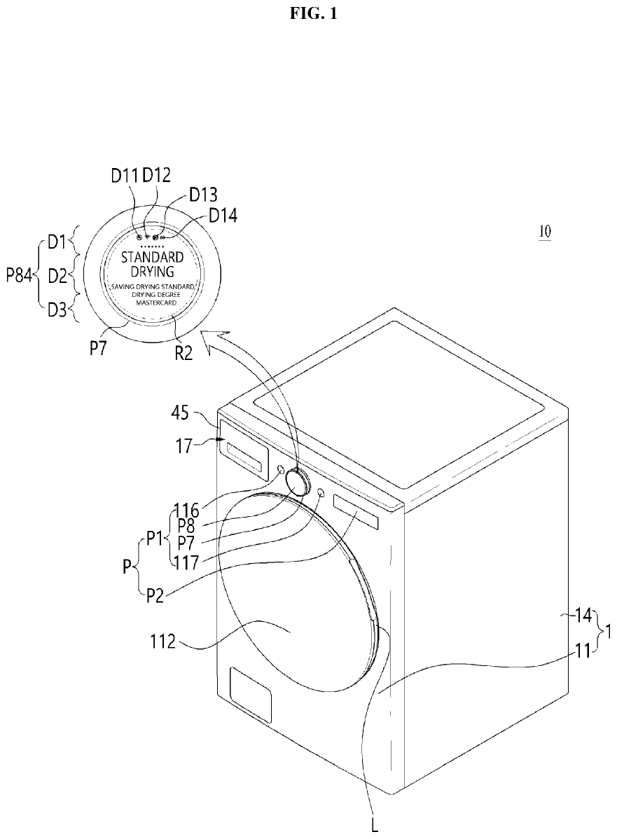

FIG. 1 shows an appearance of a laundry treating apparatus of the present disclosure.

As shown in FIG. 1, a laundry washing apparatus 10 includes a cabinet 1 and a control panel P disposed on the cabinet 1.

The control panel P described in the present application refers to a device or a program that enables communication between a user and the laundry treating apparatus (e.g., including other electronic devices in addition to the laundry treating apparatus). The communication between the user and the laundry treating apparatus means a process in which the user inputs a control command to the laundry treating apparatus and a process in which the laundry treating apparatus transmits information to the user.

The user may input, via the control panel P, at least one of a power command of supplying or cutting off the power of the laundry treating apparatus, a selection command of selecting the arbitrary course or option that treats the laundry, an execution command of performing the selected course or option, and a stop command of stopping the course or option being performed.

The treatment of the laundry may include the washing cycle of removing the foreign substances of the laundry via water and the detergent, or a drying cycle of drying water contained in the laundry.

The control panel P may display a state or information of the laundry treating apparatus to the user. For example, the control panel P may display a state in which at least one of the power command, the selection command, the execution command, the performance command, and the stop command is input. In addition, the control panel P may display error information of displaying a problem situation occurring in the laundry treating apparatus, or guide information of guiding an action to be taken by the user.

The cabinet 1 may include the front panel 11 forming a front face of the cabinet 1, and the top panel 13 coupled to a top face of the front panel 11. The front panel 11 and the top panel 13 may be made of a metal material, and may have a steel plate shape.

The control panel P of the laundry treating apparatus of the present disclosure may be coupled to the front panel 11.

The control panel P may be coupled to a rear face of the front panel 11 and may be partially exposed to a front face of the front panel 11. The front panel 11 may be manufactured as a simple metal plate, and the control panel P may be simply coupled to the rear face of the front panel 11 and be fixed.

Accordingly, the front panel 11 may form a most area of the front face of the cabinet 1. As a result, a sense of unity of the front panel 11 may be strengthened and an aesthetics sensation may be maximized. In addition, a process of manufacturing the front panel 11 may be simplified, and a process of assembling or installing the front panel 11 and the control panel P may be simplified. In addition, a separate component such as a frame for mounting the control panel P on the front panel 11 may be omitted.

The control panel P of the laundry treating apparatus of the present disclosure may include a manipulation portion P7 rotatably coupled to the cabinet, and a display portion P8 disposed inside the manipulation portion P7 to display the state of the laundry treating apparatus.

The manipulation portion P7 may be formed in a shape of a rotary knob, and the display portion P8 may be formed as a display D including a liquid crystal or the like. It may be seen that the display portion P8 and a selection portion R to be described later are included in the manipulation portion P7.

The display portion P8 may be entirely formed as a touch panel, or may be at least partially formed as the touch panel.

The manipulation portion P7 may be configured to rotate to select the arbitrary course or option that may treat the laundry, and the display portion P8 may be configured to display a corresponding course or option each time the manipulation portion P7 rotates.

As a result, the control panel P of the laundry treating apparatus of the present disclosure may prevent an arbitrary course or option capable of rotating the drum to be described later from being displayed as characters, guide phrases, and the like in a certain area. Accordingly, an area or an area ratio occupied by the control panel P in the front panel 11 may be greatly reduced, and the aesthetics sensation may be maximized as no separate characters or guide phrases are attached to the front panel 11.

A display P84 of the display portion P8 may include a state display area D1 for displaying the state of the laundry treating apparatus, and a content display area D2 for displaying the state in which the at least one of the power command, the selection command, the execution command, the performance command, and the stop command of the laundry treating apparatus is input, the error information, or the guide information of guiding the action to be taken by the user.

In addition, the control panel P may further include the selection portion R for the user to input a determination command of determining the information to be displayed on the display portion P8. The selection portion R may be disposed inside the manipulation portion P7 to further reduce an area occupied by the control panel P.

The selection portion R may be disposed inside the display portion P8 and may be disposed spaced apart from the content display area D2. The selection portion R may be formed as a physical button or a touch panel for sensing a contact of a user's body.

The display P84 of the display portion may include an input area D3 that may request an input of the selection portion R at a point corresponding to the selection portion R.

The input area D3 may be configured to emit light when the selection portion R may be activated to receive the command from the user.

In one example, the manipulation portion P7 and the display portion P8 may be operated only when the power is supplied.

To this end, the control panel P of the laundry treating apparatus of the present disclosure may further include the power switch P46 for inputting the power command to the laundry treating apparatus, as well as the manipulation portion P7 and the displaying interface.

The user may activate the manipulation portion P7 by inputting the power switch P46.

The front panel 11 may include a power switch mounting portion 116 formed such that the power switch P46 may be installed.

In addition, the control panel P may further include the executing means P47 to which the performance command of performing or stopping the selected course or option is input. The executing means P47 may be formed separately from the manipulation portion P7 and the display portion P8 to reflect a definite performance or stop intention of the user. In addition, the functions of the manipulation portion P7 and the display portion P8 may be prevented from being excessive.

The front panel 11 may include an executing means mounting portion 117 formed such that the executing means P47 may be installed.

The power switch P46, the execution device P47, the manipulation portion P7, and the display portion P8 may be arranged in one control P1.

In one example, the control panel P may further include the setting portion P2 that may add an option to the course or change the option of the course on the front panel 11. The user may set an option capable of changing an intensity, a duration, and the like of the course via the setting portion P2.

The setting portion P2 may be formed separately from the casing P1 and may be coupled to the front panel 11. The setting portion P2 may include a separate liquid crystal and may include a touch panel or a physical button to input the option.

In one example, the front panel 11 may have a door 112 that opens and closes the laundry inlet 111 through which the laundry is put into the laundry treating apparatus. The control panel P may be located above the door 112 to enhance accessibility of the user.

In one example, the front panel 11 may further include locking means L for fixing the door 112 to the front panel 11. When the laundry treating apparatus operates, such as the drum of the laundry treating apparatus rotates, the locking means L may lock the door 112 to the front panel 11. Therefore, a safety accident may be prevented. The locking means L may unlock the door 112 when the operation of the laundry treating apparatus is completed.

The locking means L may be formed as any component as long as it is able to fix the door 112 to the cabinet 1. The locking means L may be formed as a fastener for fastening a hook protruding from the door, or may be formed as a solenoid valve for holding the hook.

The laundry treating apparatus of the present disclosure may include a detergent box 45 that accommodates therein the detergent for washing the laundry, and the front panel 11 may include a detergent hole 17 from which the detergent box 45 is extended.

The cabinet 1 includes the front panel 11 forming the front face of the laundry treating apparatus. The front panel 11 may include the laundry inlet 111 defined to be in communication with the drum 2, and the door 112 pivotably coupled to the cabinet to open and close the laundry inlet 111.

The control panel P may be disposed on the front panel 11. As a result, the control panel P may include the input means P9 that receives the control command from the user, and the display portion P8 that outputs the information such as the control command selectable by the user. The main controller M1 for controlling a command for performing the drying operation of the laundry treating apparatus may be installed.

The input means P9 may include a power supply requesting device P46 for requesting the power supply to the laundry treating apparatus, the manipulation portion P7 that enables the user to select a desired course among multiple courses, and the executing means P47 for requesting start of the course selected by the user.

The display portion P8 may include at least one of a display panel capable of outputting text and figures, and a speaker capable of outputting a voice signal and sound.

In one example, the laundry treating apparatus of the present disclosure may include a water storage 7 constructed to separately store moisture generated in the process of drying the laundry. The water storage 7 may include a water storage tank 72 constructed to be extended from one side of the front panel 11 to the outside. The water storage tank 72 may be constructed to collect condensed water received from a washing pump to be described later.

The user may extend the water storage tank 72 from the cabinet 1, then remove the condensed water, and then re-mount the water storage tank 72 in the cabinet 1. As such, the laundry treating apparatus of the present disclosure may be disposed at any place where a sewer or the like is not installed.

The cabinet 1 may include a tank hole 115 through which the water storage tank 72 may be extended to the outside. The tank hole 115 may be defined in the front panel 11.

Accordingly, the water storage tank 72 may be retracted into and extended from the front panel 11 through the tank hole 115 in a slide manner. As a result, even when the water storage tank 72 has a large volume to store therein a larger amount of condensed water, the user may easily extend the water storage tank 72 from the front panel 11. That is, the user may extend the water storage tank 72 from the cabinet 1 without overcoming a weight of the water storage tank 72 by himself/herself.

The tank hole 115 may be defined at a position where the water storage tank 72 does not interfere with the control panel 117 when the water storage tank 72 is extended to the outside. For example, the tank hole 115 may be defined on one side of the control panel 117.

In one example, the tank hole 115 may be defined above the door 112. Accordingly, when the user extends the water storage tank 72 from the front panel 11, the user may bend a waist relatively less.

As a result, the water tank 430 and the water storage tank 72 may be disposed above a rotation center of the drum.

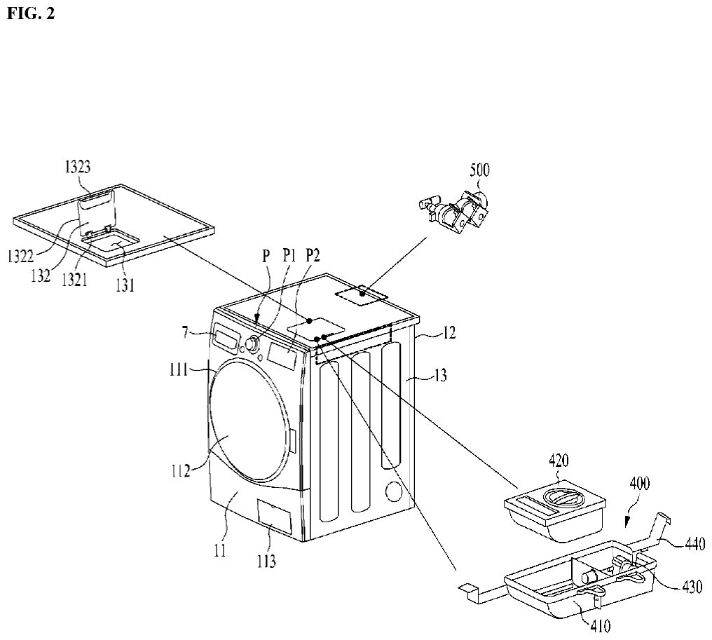

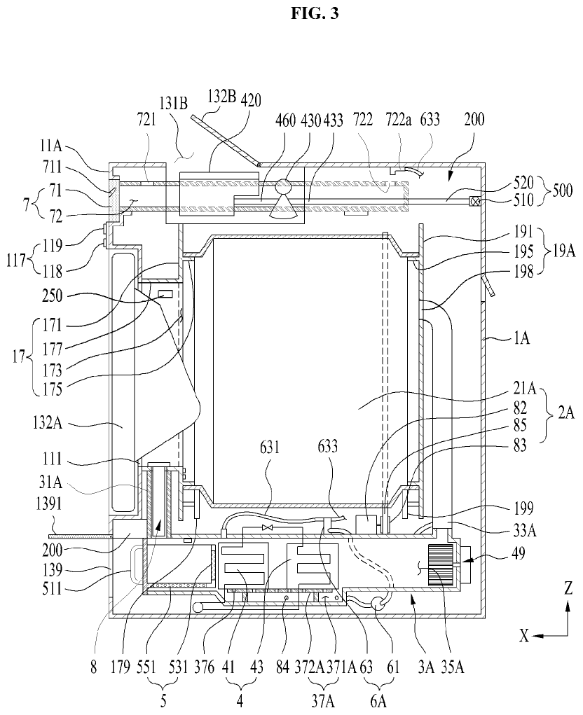

FIG. 2 shows a basic structure of a laundry treating apparatus according to the present disclosure.

The cabinet 1 may include a front panel 11 defining a front surface of the laundry treating apparatus, and the front panel 11 may include an inlet 111 connected to a drum 2, and a door 112 rotatably coupled to the cabinet and configured to open and close the inlet 111.

The front panel 11 may include a control panel P, the control panel 117 may include an input means P9 configured to receive a control command from a user, and a display portion P8 configured to output information such as a control command to be selected by the user, and a main controller M2 configured to control a command for executing a drying cycle of the laundry treating apparatus may be installed.

The input means P9 may include a power switch P46 requesting power supply to the laundry treating apparatus, a manipulation portion P7 for allowing the user to select a desired course among multiple courses, and an executing portion P47 requesting start of the course selected by the user.

The display portion P8 may include at least one of a display panel for outputting a text and a figure, or a speaker for outputting a voice signal and sound.

The laundry treating apparatus according to the present disclosure may include a water storage 7 configured to separately store moisture generated in a process of drying the laundry. The water storage 7 may include a water storage tank 72 configured to taken out to the outside from one side of the front panel 11. The water storage tank 72 may be provided to collect condensate received from a washing pump to be described later.

The user may draw out the water storage tank 72 from the cabinet 1, may remove the condensate, and may then mount the water storage tank 72 in the cabinet 1 again. Accordingly, the laundry treating apparatus according to the present disclosure may be disposed at any place in which a sewer or the like is not installed.

The cabinet 1 may include a tank hole 115 from which the water storage tank 72 is drawn out. The tank hole 115 may be included in the front panel 11.

Thus, the water storage tank 72 may be slid in and out of the front panel 11 through the tank hole 115. As a result, even if the water storage tank 72 has a large volume and stores a larger amount of condensate, the user may easily draw out the water storage tank 72 from the front panel 11. That is, the user may draw out the water storage tank 72 from the cabinet 1 without autonomously overcoming the weight of the water storage tank 72.

When the control panel 117 and the water storage tank 72 are drawn in and out, the tank hole 115 may be placed at a position at which the water storage tank 72 does not interfere with the control panel 117. For example, the tank hole 115 may be provided at one side of the control panel 117.

The tank hole 115 may be disposed on the upper portion of the door 112. Accordingly, when the user draws out the water storage tank 72 from the front panel 11, the user may bend the waist relatively less.

As a result, a water tank 430 and the water storage tank 72 may be disposed above the center of rotation of the drum.

The laundry treating apparatus according to the present disclosure may further include a steam part 200 for supplying steam to the inside of the laundry or the cabinet. The steam part 200 may be provided to generate steam by receiving fresh water rather than condensate. The steam part 200 may be provided to generate steam by heating water, using ultrasonic waves, or vaporizing water.

The steam part 200 is provided to generate steam by receiving a certain amount of water, and thus may occupy a certain volume. In this case, the front panel 11 of the cabinet may be equipped with a door and the control panel 117, and a rear panel 12 of the cabinet may be equipped with a duct and a water supply for supplying or discharging air to or from the drum, and thus the steam part 200 may be installed on a side panel 14 of the cabinet.

The laundry treating apparatus according to the present disclosure may include a steam controller 800 configured to separately control the steam part 200. Although the steam controller 800 is installed on the control panel 117, a separate control panel may be provided to prevent overload of the control panel 117 and to prevent increase in production cost.

The steam controller 800 may be provided adjacent to the steam part 200. The steam controller 800 may be provided on the side panel 14 on which the steam part 200 is installed to reduce the length of a control line connected to the steam part 200.

Since the steam part 200 supplies steam to be in contact with the laundry, the steam part 200 may generate steam with fresh water. Since water collected in the water storage 7 is generated in laundry, the collected water may be likely to contain lint or foreign substances and may not be suitable for generating the steam.

Accordingly, the laundry treating apparatus according to the present disclosure may include a water supply 300 configured to supply water to the steam part 200 and separately provided from the water storage 7. The water supply 300 may store fresh water therein for may be provided to receive fresh water from the outside and to supply the fresh water to the steam part 200.

For example, the water supply 300 may include an external supply 500 configured to receive water from an external water supply source and to transmit water to the steam part 200, and an internal supply 400 configured to separately store fresh water and to supply the fresh water to the steam part 200.

The internal supply 400 may further include a water tank 420 that is separately provided from the water storage 7 to store fresh water, a water pump 420 configured to supply water of the water tank 420 to the steam part 200, and a tank housing 410 that accommodates the water tank 420 and the water pump 420 inside the cabinet.

The laundry treating apparatus according to the present disclosure may be configured to supply water of the water tank 420 to the steam part 200 using the weight of the water based on a difference in an installation height between the water tank 420 and the steam part 200.

When the difference in the installation height between the water tank 420 and the steam part 200 is not ensured, the water pump 420 may be further installed. When the water pump 420 is further installed, the space inside the cabinet 1 may be advantageously used more densely.

The external supply 500 may include a direct water valve 520 connected to the external water supply source to receive water.

The laundry treating apparatus according to the present disclosure may further include a determiner 700 configured to determine which one of the external supply 500 and the internal supply 400 is to be preferentially used to supply water to the steam part 200.

The determiner 700 may be provided to determine which one of the external supply 500 and the internal supply 400 is to be used preferentially.

The water tank 420 may be configured to store fresh water. The water tank 420 may be exposed out of the cabinet 1 to frequently refill fresh water.

The water tank 420 may be drawn out from the cabinet 1. Thus, the user may draw out the water tank 420 from the cabinet 1 to simply refill water.

The water tank 420 may be drawn out through the front panel 11. However, when the water storage tank 72 is drawn out from the front panel 11, it may also be difficult to ensure a region for drawing out the water tank 420 in the front panel 11 due to an area occupied by the control panel P.

Thus, the water tank 420 may be drawn out from a top panel 13 to be prevented from interfering with the control panel 117. The water tank 420 may store water and the water storage 7 may also store water, and thus the user may confuse them. To this end, the laundry treating apparatus according to the present disclosure may be provided in different directions or positions in which the water tank 420 and the water storage 7 are exposed in the cabinet.

Thus, the water tank 420 may be exposed out of the top panel 13, and the water storage 7 may be exposed out of the front panel 11. Thus, although both the water tank 420 and the water storage 7 are provided, the user may be prevented from confusing the water tank 420 and the water storage 7. The water tank 420 needs to store fresh water and to maintain the freshness of stored water, and thus may have a smaller volume than the water storage 7. Thus, the user may distinguish the water tank 420 and the water storage 7 due to the volume difference.

Since the water tank 420 has a smaller volume than the water storage 7, the water tank 420 may be easily drawn out upward. Thus, the water tank 420 may be drawn out upward from the top panel 13. As a result, the water tank 420 and the water storage 7 may be drawn out in different directions, and the possibility of user confusion may be further reduced.

The top panel 13 of the laundry treating apparatus according to the present disclosure may include a tank draw-out hole or draw-out hole 131 through which the water tank 420 is exposed to the outside or is exposed out of the cabinet. The tank draw-out hole 131 may correspond to a cross-sectional area of the water tank 420 or may be provided slightly larger than the area of the water tank 420.

The top panel 13 may further include a draw-out cover 132 configured to shield the tank draw-out hole 131 and to prevent the water tank 420 from being arbitrarily drawn out. The draw-out cover 132 may include one or more panel couplers 1321 to be coupled to an outer circumference of the tank draw-out hole 131. The panel coupler 1321 may extend from one side of the draw-out cover 132 to rotatably couple the draw-out cover 132 to the top panel 13. The panel coupler 1321 and the top panel 13 may be hinged to each other.

The draw-out cover 132 may include a panel handle 1323 to be gripped by the user on a surface, and the panel handle 1323 may be provided as a groove concavely downward from the draw-out cover 132. The draw-out cover 132 may further include a panel fixer 1332 detachably coupled to an outer circumference of the top panel 13 or the tank draw-out hole 131. The panel fixer 1332 may be coupled to the top panel 13 or the tank draw-out hole 131 in a push-button manner.

The laundry treating apparatus according to the present disclosure may further include a filter for removing foreign substances from circulated air. The front panel 11 may include a filter mounting hole 113 provided to allow the filter to be drawn out or being inserted.

FIG. 3 shows a structure of the laundry drying apparatus 20 of the laundry treating apparatus of the present disclosure.

The laundry drying apparatus 20 may include a drying cabinet 1A, a drying drum 2A that is rotatably disposed inside the drying cabinet to provide a space for storing the laundry, a circulation flow path 3A that forms a flow path for re-supplying air discharged from the drying drum 2 to the drying drum 2A, and a heat exchanger 4 that dehumidifies and heats air introduced into the circulation flow path 3A and then re-supplies dehumidified and heated air to the drying drum 2A.

When the drying drum 2A is formed as a cylindrical drum body 21A with open front face and rear face, a first support 17 that rotatably supports the front face of the drying drum 2A and a second support 19A that rotatably supports the rear face of drying drum 2A may be disposed inside the cabinet 1A.

The first support 17 may include a first fixed body 171 fixed inside the drying cabinet 1A, a drum laundry inlet 173 defined to extend through the first fixed body to allow the laundry inlet 111 and an interior of the drum body 21A to be in communication with each other, and a first support body 175 disposed on the first fixed body 171 and inserted into a front face (e.g., a first open face) of the drum body 21A.

The first fixed body 171 may be in any shape as long as the drum laundry inlet 173 and the first support body 175 are able to be disposed. The first support body 175 may be formed in a pipe shape protruding from the first fixed body 171 toward the drying drum body 21A. A diameter of the first support body 175 may be larger than a diameter of the drum laundry inlet 173, and may be smaller than a diameter of a front face of the drying drum body 21A. In this case, the drum laundry inlet 173 will be located inside the space defined by the first support body 175.

The first support 17 may further include a connection body 177 that connects the laundry inlet 111 and the drum laundry inlet 173 to each other. The connection body 177 may be formed in a pipe shape extending from the drum laundry inlet 173 toward the laundry inlet 111. The connection body 177 may have an air outlet 178 in communication with the circulation flow path 3A.

The air outlet 178, as a passage that allows air inside the drying drum body 21A to flow to the circulation flow path 3, may be defined as a through-hole defined to extend through the connection body 177.

The second support 19A may include a second fixed body 191 fixed inside the cabinet 1A, and a second support body 195 disposed on the second fixed body 191 and inserted into a rear face (e.g., a second open face) of the drum body 21A. The second support 19 includes an air inlet 198 defined to extend through the second fixed body 191 to allow an interior of the drum body 21 and an interior of the drying cabinet 1A to communicate with each other. In this case, a duct 3A may be constructed to connect the air outlet 178 and the air inlet 198 to each other.

The cylindrical drum body 21A with an empty interior may be rotated by various types of drying drivers. A case in which the drying driver 82,83,85 includes a motor 82 fixed inside the drying cabinet 1A, a pulley 85 rotated by the motor, and a belt 83 that connects a circumferential face of the pulley 85 and a circumferential face of the drum body 21A to each other is shown as an example.

In one example, the drying driver may be disposed on a rear face of the drying drum 2A to directly rotate the drum 2A. A rotation shaft of the drying driver 82,83,85 may be directly coupled to the drum 2A to rotate the drying drum 2A.

In this case, the second support 19A may support the drying driver 82,83,85 to be located on a rear face or at a center of the drying drum 2A.

In one example, a separate reducer may be disposed between the drying driver 82,83,85 and the drying drum 2A. In this case, a rotation shaft from the reducer may be directly coupled to a center of the rear face of the drum 2A, and the reducer may be coupled to and supported by the second support 19.

In this regard, the drying driver 82,83,85 may freely change a rotation speed and a rotation direction of the drum 2A.

The first support 17 may have a first roller 179 that rotatably supports the circumferential face of the drum body 21A, and the second support 19 may have a second roller 199 that rotatably supports the circumferential face of the drum body.

The circulation flow path 3A may include a duct in communication with the drum 2. The duct 3A may be in communication with the drying drum 2A, so that it may be considered that the duct 3A forms a circulation flow path in which air discharged from the drying drum passes the heat exchanger 4 and is re-introduced into the drum 2A.

The duct 3A may include an exhaust duct 31A connected to the air outlet 178, a supply duct 33A connected to the air inlet 198, and a connection duct 35A that connects the exhaust duct and the supply duct to each other.

The heat exchanger 4 may be formed as various apparatuses as long as the dehumidification and the heating of air introduced into the duct 3A may be sequentially performed. For example, the heat exchanger 4 may be formed as a heat pump system.

The heat exchanger 4 may include a fan 49 that allows air to flow along the duct 3A, a first heat exchanger (e.g., a heat absorber) 41 that removes moisture from air introduced into the duct 3A, and a second heat exchanger (e.g., a heater) 43 that is disposed inside the duct 3A to heat air that has passed through the first heat exchanger 41.

The heat absorber 41 may be formed as an evaporator that absorbs heat, and the heater 43 may be formed as a condenser that emits heat.

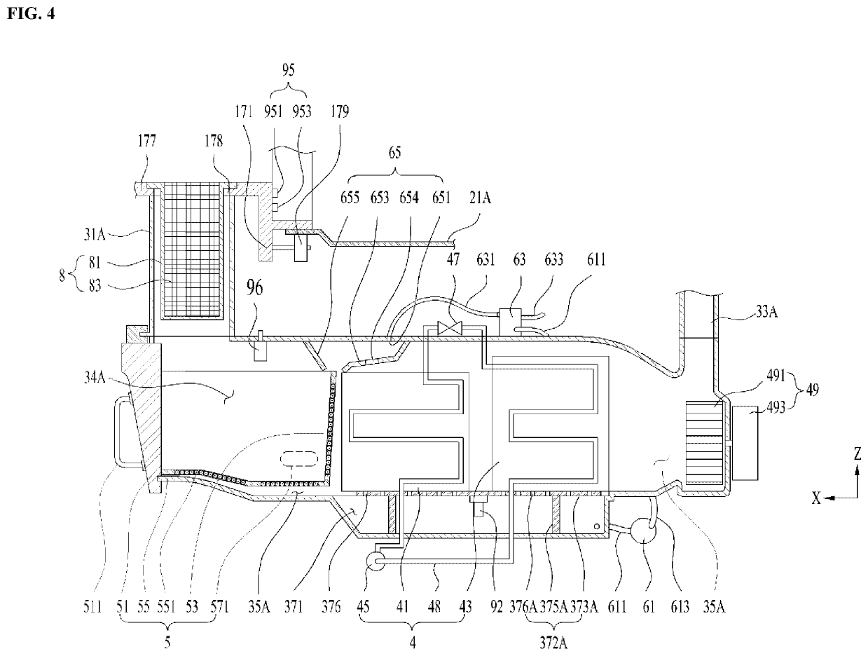

The fan 49 may include an impeller 491 disposed inside the duct 3A, and an impeller motor 493 that rotates the impeller 491 (see FIG. 4). The impeller 491 may be disposed in any of the exhaust duct 31A, the connection duct 35A, and the supply duct 33A. The impeller 491 may be disposed in the supply duct 33A.

The heat absorber 41 is formed with multiple metal plates disposed along a width direction (e.g., a Y-axis direction) of the connection duct 35A or a height direction (e.g., a Z-axis direction) of the connection duct, and the heater 43 may be formed with multiple metal plates disposed along the width direction of the connection duct or the height direction of the connection duct. The heat absorber 41 and the heater 43 are sequentially arranged inside the connection duct 35A in a direction from the exhaust duct 31A toward the supply duct 33A, and are connected to each other via a refrigerant pipe 48 that forms a circulation flow path of a refrigerant.

The refrigerant moves along the refrigerant pipe 48 by a compressor 45 located outside the duct 3A, and the refrigerant pipe 48 has a pressure regulator 47 that adjusts a pressure of the refrigerant that has passed through the heater 43.

The heat absorber 41 is means for cooling air and evaporating the refrigerant by transferring the heat of air introduced into the exhaust duct 31A to the refrigerant. The heater 43 is means for heating air and condensing the refrigerant by transferring the heat of the refrigerant that has passed through the compressor 45 to air. In this case, the moisture contained in air will be collected on a bottom face of the connection duct 35A along a surface of the heat absorber 41 when passing through the heat absorber 41.

In order to collect water removed from air passing through the heat absorber 41, the laundry drying apparatus 20 has a water collecting portion.

The water collected in the water collecting portion may be collected in a water storage 7 and discharged all at once later. The water storage 7 may include a water storage tank 72 that is detachably disposed in the cabinet 1 to provide a space for storing water, and an inlet 722 defined to extend through the water storage tank 72 to introduce water discharged from a water storage supply pipe 633 into the water storage tank 72.

The water storage tank 72 may be formed as a tank in a form of a drawer extended from the cabinet 1. In this case, the front panel 11A of the cabinet must have a water storage mounting hole or tank hole into which the water storage tank 72 is inserted. A water storage panel 71 is fixed to a front face of the water storage tank 72. The water storage panel 71 may be detachably coupled to the water storage mounting hole or tank hole 115 to form a portion of the front panel 11A.

The water storage panel 71 may further include a groove 711 into which a user's hand is inserted. In this case, the water storage panel 71 will also perform a function of a handle for extending or retracting the water storage tank 72 from or into the cabinet.

The inlet 722 may be defined to receive water discharged from a nozzle 722a fixed to the drying cabinet 1A. The nozzle 722a may be fixed to a top panel 13 of the cabinet so as to be positioned above the inlet 722 when the storage body 72 is inserted into the cabinet 1.

In the water storage 7 having the above structure, the user may discard water inside the water storage tank 72 by extending the water storage tank 72 from the cabinet 1 and then inverting or tilting the water storage tank 72 in a direction in which the inlet 722 is directed. A communication hole 721 defined to extend through a top face of the storage body 72 may be further defined such that water inside the water storage tank 72 is easily discharged through the inlet 722.

In one example, the heat exchanger 4 is constructed to condense the moisture in air circulating in the heat absorber 41. Therefore, even when the air circulates in the drum 2A, the moisture is removed by the heat absorber 41, so that the laundry inside the drum 2A may be continuously dried.

The moisture condensed in the heat absorber 41 may be first collected in the water collecting portion 37A and then collected secondary in the water storage 7. The water collecting portion 37A may be located inside the connection duct 35, and may be separately disposed in a space spaced apart from the connection duct 35.

FIG. 4 shows a detailed structure of the water collecting portion 37A, the heat exchanger 4, and a washer 6.

The water collecting portion 37A may be formed as a water collecting body 371A fixed to a bottom face of the connection duct 35A and in communication with the interior of the connection duct. A heat exchanger support 372A may be further disposed inside the water collecting body 371A such that the heat absorber 41 and the heater 43 do not come into contact with water (e.g., condensed water) stored in the water collecting body 371A. The heat exchanger support 372A may include a support plate 373A in contact with the heat absorber 41 and the heater 43, a spacer 375A that maintains a gap between the support plate 373 and a bottom face of the water collecting body 371A, and a support plate through-hole 376A defined to extend through the support plate 373A.

The support plate through-hole 376A may be defined only in a space in which the heat absorber 41 is supported of the space provided by the support plate 373A, and may be defined in each of the space in which the heat absorber is supported and a space in which the heater is supported. When the support plate through-hole 376A is also defined in a bottom face of the heater 43, water flowed to the heater 43 along the support plate 373A may be discharged to the water collecting portion 371A (e.g., to prevent a decrease in heat transfer efficiency that occurs when the heater comes into contact water).

In order to minimize the foreign substances (e.g., lint and the like) discharged from the drying drum body 21A being stacked in the heat absorber 41 and the heater 43, the laundry drying apparatus 20 may further include filtration means for filtering air.

Second filtration means 8 may be formed as means for filtering air introduced into the exhaust duct 31A from the drying drum body 21A, and first filtration means 5 may be located between the second filtration means 8 and the heat absorber 41 and be formed as means for filtering air that has passed through the second filtration means. A diameter of a filtration hole defined in the first filtration means 5 may be set smaller than a diameter of a filtration hole defined in the second filtration means 8.

The second filtration means 8 may include a frame 81 that is detachably inserted into the exhaust duct 31A through the air outlet 178, and a filter (e.g., a fourth filter) 83 disposed in the frame to filter air.

The first filtration means 5 may be detachably disposed in the connection duct 35. In this case, the front panel 11 of the cabinet may have a filter mounting hole from which the first filtration means 5 is withdrawn and a mounting hole door that opens and closes the filter mounting hole, and the duct 3A may have a duct through-hole 34 (see FIG. 3) into which the first filtration means 5 is inserted. Accordingly, the user may remove the foreign substances remaining in the first filtration means 5 and wash the first filtration means after separating the first filtration means 5 from the laundry treating apparatus as needed.

The first filtration means 5 may include a filtration means body 51, 53, and 55 inserted into the filter mounting hole and the duct through-hole 34 and positioned between the second filtration means 8 and the heat absorber 41, and filters 531, 551, and 571 disposed in the filtration means body to filter fluid (e.g., air and water) flowing to the heat absorber 41 and the water collecting portion 371A.

The filtration means body may be formed in various forms depending on a shape of a cross-section (e.g., a Y-Z plane and a X-Z plane) of the connection duct 35A. FIG. 1 shows an example of a case in which the filtration means body is formed in a shape similar to a hexahedron.

In this case, the filtration means body may include a front face 51 formed in a shape capable of closing the duct through-hole 34, a rear face 53 positioned between the front face and the heat absorber 41, a bottom face 55 constructed to connect the front face and the rear face with each other, and a first side face 57 and a second side face 58 that form a left side face and a right side face of the filtration means body.

The front face 51 may have a lock 511 removably coupled to a lock fastening portion 16 disposed in the cabinet. The lock 511 may be formed as a bar rotatably coupled to the front face 51 of the filtration means body, and the lock fastening portion may define a groove in which a free end of the bar is accommodated. Preferably, the locks 511 are respectively disposed on opposite sides of the front face 51, and the lock fastening portions 16 are respectively disposed on opposite sides of the filter mounting hole.

To facilitate insertion of the filtration means body into the connection duct 35A or removal of the filtration means from the connection duct 35A, the front face 51 may further include a handle 511.

The rear face 53 and the bottom face 55 may respectively have a first filter 531 and a second filter 551 for filtering the fluid (e.g., air and water) introduced into the filtration means body. The rear face 53 has a rear face through-hole defined therein for allowing an interior of the filtration means body and the internal space of the duct 3A to be in communication with each other, and the first filter 531 is disposed in the rear face through-hole. The bottom face 55 has a bottom face through-hole for allowing the interior of the filtration means body and the internal space of the duct 3A to be in communication with each other, and the second filter 551 is disposed in the bottom face through-hole. Therefore, the first filter 531 is means for filtering the fluid (e.g., air and water) supplied to the heat absorber 41, and the second filter 551 is means for filtering the fluid supplied to the water collecting portion 371A.

The first side face 57 and the second side face 58 may be constructed to connect the front face 51, the rear face 53, and the bottom face 55 to each other.

The first filtration means 5 having the above structure may be in communication with the exhaust duct 31A through the top face or the second side face 58 of the filtration means body. FIG. 1 shows a case in which the first filtration means 5 is connected to the exhaust duct 31A through a top face through-hole defined to extend through the top face of the filtration means body and a side face through-hole defined to extend through the second side face 58 as an example.

The first filter 531 may be inclined in an angle range from 90 degrees to 100 degrees toward a front face of the heat absorber 41 based on the bottom face 55 of the filtration means body. This is to allow the foreign substances remaining in the first filter to easily move to the bottom face 55 when water is injected into the first filter 531 via the washer 6 to be described later.

The second filter 551 may be inclined downward in an angle range from 10 to 20 degrees from the front face 51 toward the first filter 531 (e.g., the second filter may be inclined upward in an angle range from 10 to 20 degrees in a direction from a lower end of the first filter to the filter mounting hole). When the second filter 551 is inclined downward toward the first filter 531, because a connection point of the first filter 531 and the second filter 551 will be the lowest point in the space provided by the first filtration means, the foreign substances of the first filtration means 5 may be concentrated at the connection point of the first filter 531 and the second filter 551. When the foreign substances are concentrated at the connection point of the first filter 531 and the second filter 551, the user will be able to more easily remove the foreign substances inside the first filtration means 5.

However, when the foreign substances are concentrated at the connection point of the first filter 531 and the second filter 551, there may be a situation in which a long time is required for water sprayed via the washer 6 to be discharged to the water collecting portion 371. In order to solve such problem, a bypass hole for allowing the interior of the first filtration means 5 and the water collecting portion 371 to be in communication with each other, and a third filter 571 disposed in the bypass hole may be further disposed.

The bypass hole and the third filter 571 may be defined at a position higher than an uppermost end of the second filter 551 and lower than an uppermost end of the first filter 531. Therefore, the laundry treating apparatus may minimize a phenomenon in which water sprayed to the first filtration means 5 is not able to be recovered at the water collecting portion 371 by the foreign substances remaining in the first filtration means 5.

In one example, the laundry drying apparatus 20 may further include the washer 6 that washes the first filtration means 5 using water stored in the water collecting portion 371A. That is, water stored in the water collecting portion 371A may be separately collected into the water storage 7 or may be selectively flowed to the washer 6.

The washer 6 may be formed as means for washing at least one of the first filter 531, the second filter 551, the third filter 571, and the heat absorbers 41 by spraying water stored in the water collecting portion 371A to the first filtration means 5. The washer 6 may include a spray 65 disposed in the duct 3A to supply water to the first filtration means 5, and a washing pump 61 that allows water stored in the water collecting portion 371A to flow to the spray 65.

The washing pump 61 may be connected to the water collecting portion 371A via a first connection pipe 611, and may be connected to the spray 65 via a second connection pipe 613. When the laundry treating apparatus is constructed to allow water of the water collecting portion 371A to flow to the spraying portion 65 and the water storage 7 with only one washing pump 61, the laundry drying apparatus 20 may further include flow path switching portion 63. In this case, the flow path switching portion 63 may be connected to the washing pump 61 via he second connection pipe 613, the spray 65 may be connected to the flow path switching portion 63 via a spray supply pipe 631, and the water storage 7 may be connected to the flow path switching portion 63 via the water storage supply pipe 633.

In this case, the water storage supply pipe 633 should connect the nozzle 722a and the flow path switching portion 63 to each other.

The flow path switching portion 63 includes a valve for controlling opening and closing of the spray supply pipe 631 and opening and closing of the water storage supply pipe 633. Therefore, the laundry drying apparatus 20 may control the valve disposed in the flow path switching portion 63 to supply water stored in the water collecting portion 371A to the spray 65 or to the water storage 7.

A case in which the spray 65 includes a duct through-hole 651 defined to extent through the connection duct 35 and to which the spray supply pipe 631 is connected, a first guide 653 that guides water supplied from the duct through-hole to the first filter 531, and a second guide 655 that guides at least a portion of water supplied through the first guide 653 to the front face of the heat absorber 41 is shown as an example. In this case, the second guide 655 may be disposed as means for allowing water to be supplied to the front face of the heat absorber 41 via the first filter 531. That is, the first filter 531 may be disposed between the first guide 653 and the second guide 655 when the first filtration means 5 is fixed to the connection duct 35A, and the second guide 655 may be formed as an inclined face inclined downward toward the first filter 531 from a top face of the connection duct 35.

The first guide 653 may further include a guide through-hole 654. The guide through-hole 654, as a hole defined to extend through the first guide 653, may allow water introduced into the duct through-hole 651 to be supplied to a front area of the heat absorber 41 through the guide through-hole 654. The front area of the heat absorber means an area positioned at a side facing toward the first filter 531 based on a vertical line passing through a center of the heat absorber 41.

In one example, it is preferable that the laundry treating apparatus of the present disclosure includes water collecting portion water level sensing means 92 that measures a water level of the water collecting portion 371A and transmits the water level to the controller. When the water collecting portion water level sensing means 92 is disposed, the laundry treating apparatus may determine a time point of allowing water stored in the water collecting portion 371A to flow to the storage body 72. Therefore, water of the water collecting portion 371A may be prevented from flowing back into the connection duct 35A.

The water collecting portion water level sensing means 92 may be formed as any device capable of sensing the water level inside the water collecting portion 371A. FIG. 3 shows a sensor with multiple electrodes having different lengths (e.g., multiple electrodes electrically connected based on the water level) as an example.

The laundry drying apparatus 20 may have dryness sensing means to determine a time point to stop operation of the heat exchanger 4 by determining a dryness of the laundry. The dryness sensing means may be formed as at least one of an electrode sensor 95 constructed to be in contact with the laundry to measure an amount of moisture contained in the laundry, and a humidity sensor for measuring humidity of air introduced into the duct 3A from the drum 3.

The electrode sensor may include a first electrode 951 and a second electrode 953 that may be fixed to the first fixed body 171 and may be in contact with the laundry inside the drum body 21A. As the dryness increases, the amount of moisture contained in the laundry will decrease (e.g., an electrical resistance of the laundry will increase), so that the laundry drying apparatus 20 may determine the dryness of the laundry by observing an electrical resistance measured when the two electrodes 951 and 953 are connected to each other via the laundry. In one example, as the dryness of the laundry increases, the amount of moisture contained in air introduced into the duct 3A will decrease, so that the laundry drying apparatus 20 may determine the dryness of the laundry by observing the humidity of air introduced into the duct 3A via the humidity sensor.

In addition, the laundry drying apparatus 20 may further include temperature sensing means 96 for measuring a temperature of air introduced into the duct 3A. The temperature sensing means 96 may be fixed to the top face of the connection duct 35A and positioned between the first filter 531 and the second filter 551.

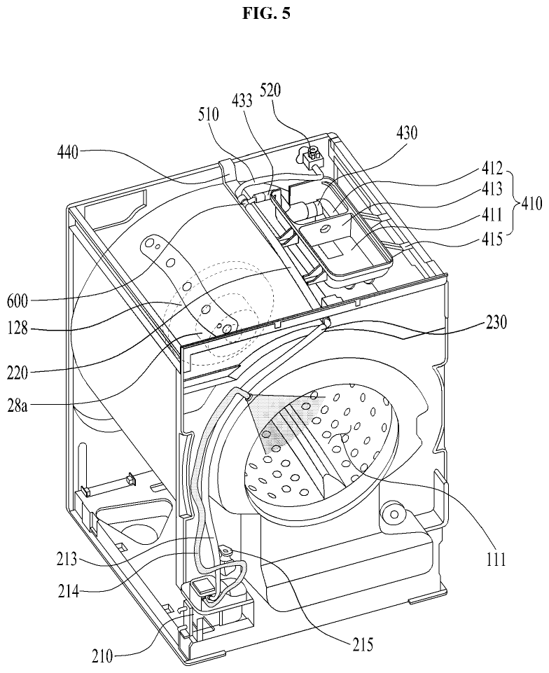

FIG. 5 shows an embodiment of an internal structure of the laundry dry apparatus 20 of the present disclosure.

The driver 28 may be directly fastened to a rear face of the drying drum 2A. The drying driver 28 may be disposed on the second support 19 to directly rotate the drying drum 2A.

The drying driver 28 may include a stator supported by the second support 19, a rotor rotated by the stator, and a rotation shaft.

The drying driver 28 may be separately coupled to a reducer 28A, and the reducer 28A may be coupled to a center (e.g., a rotation center) of a rear face of the drying drum 2A to rotate the drying drum 2A.

Accordingly, the reducer 28A may increase a torque while reducing an RPM of the rotation shaft.

The steam apparatus 200 may be disposed to be fixed to the front panel 11 or the first support supporting the drum 2 for space utilization. In addition, the steam apparatus 200 may be disposed adjacent to a corner of the cabinet 1.

The internal supply 400 may include a water tank 420 for storing water, a water pump 430 for supplying power for supplying the water stored in the water tank 420 to the steam apparatus 200, and a tank housing 410 that provides a space in which the water pump 430 and the water tank 420 are installed.

The tank housing 410 may be formed in a box shape with an open top face, and may extend along a front and rear direction of the cabinet 1 such that the water tank 420 may be disposed in a front portion of the cabinet 1 and the water pump 430 may be disposed in a rear portion of the cabinet 1.

The tank housing 410 may include a tank mounting portion 411 in which the water tank 420 is detachably mounted, and a pump mounting portion 412 in which the water pump 430 may be mounted. The tank mounting portion 411 and the pump mounting portion 412 may be formed in a recessed shape to prevent water leaking from the water tank 420 or the water pump 430 from leaking to the drying drum 2A or the like.

In addition, the tank housing 410 may further include a partition wall 413 partitioning the tank mounting portion 411 and the pump mounting portion 412 from each other. Accordingly, the water tank 420 may be easily mounted in and detached from the tank housing 410. The partition wall 413 may also serve to collect residual water in the tank mounting portion 411 or residual water in the pump mounting portion 412 such that the residual water does not flow to another place.

An extension pipe 416 for allowing the water tank 420 and the water pump 430 to be in communication with each other may be installed in the partition wall 413. A valve structure may be installed on the extension pipe 416 to prevent leakage of water even when the water tank 420 is removed from the tank mounting portion 411.

The extension pipe 416 may extend from the partition wall 413 toward the water pump 430 or toward the water tank 420.

In one example, the tank housing 410 may be formed such that the pump mounting portion 412 is disposed closer to the steam apparatus 200 than the tank mounting portion 411. Accordingly, a flow path from the water tank 420 to the steam apparatus 200 may become simplified.

The tank housing 410 may be formed such that the tank mounting portion 411 and the pump mounting portion 412 are arranged along a front and rear direction of the cabinet.

The water supply 300 may include a coupling portion capable of fixing the tank housing 410 to at least one of the support bar 440 and one side face of the cabinet.

A rotating drying drum 2A is disposed inside the drying cabinet 1. Accordingly, the components arranged inside the cabinet 1 need to be spaced apart from the drying drum 2A. Therefore, it is necessary to prevent the water supply 300 and the steam apparatus 200 from coming into contact with the drum 2. For example, a steam guide pipe 230 for supplying steam from the steam apparatus 200 to the drum 2, the direct water pipe 510 for supplying water to the steam apparatus 200, or the like needs to be blocked from coming into contact with the drying drum 2A.

It is necessary to support not only the water tank 420 and the water pump 430 but also a load of water accommodated in the water tank 420.

Therefore, the laundry treating apparatus of the present disclosure may include a support bar 440 for supporting the steam apparatus 200 and the water supply 300 to prevent the steam apparatus 200 and the water supply 300 from coming into contact with the drum, and supporting the steam apparatus 200 and the water supply 300 inside the drying cabinet.

The support bar 440 may be constructed to support at least a portion of the steam apparatus 200 or at least a portion of the water supply 300. In addition, the support bar 440 may fix or support the tank housing 410 to the drying cabinet.

The support bar 440 may be formed in a shape of a bar having both ends respectively coupled to the drying front panel 11 and the drying rear panel 12. Accordingly, the support bar 440 may support a load of the tank housing 410 as well as fix the drying front panel 11 and the drying rear panel 12. The support bar 440 may be spaced apart from the drying side panel 14 by a predetermined distance and coupled to the drying front panel 11 and the drying rear panel 12. In one example, the both ends of the support bar 440 may be coupled to the cabinet 1, but remaining portion thereof may be disposed at a lower position than an upper portion of the side face of the cabinet 1. Accordingly, the support bar 440 may be spaced apart from the top panel 13 to prevent interference with the top panel 13. In addition, a space capable of supporting some components of the water supply 300 and the steam apparatus 200 may be defined between the support bar 440 and the top panel 13.

An excessive extension of a width of the tank housing 410 may be prevented by the support bar 440.

In one example, the tank housing 410 may further include a mounting sensor capable of sensing mounting of the water tank 420 on an inner face thereof. The mounting sensor may be formed as a weight sensor, and may be configured to distinguish whether the weight is small or great.

The mounting sensor may be connected to the drying control panel PA to transmit information on whether the water tank 420 is mounted and an amount of water accommodated in the water tank 420.

The internal supply 400 may include a pump discharge pipe 433 for discharging water from the pump housing 430 to the steam apparatus 200.

The external supply 500 may include a direct water valve 520 seated on the second support 19A or the drying rear panel 12, and a direct water pipe 510 constructed to supply water from the direct water valve 520 to the steam apparatus 200.

The direct water pipe 510 may extend from the drying rear panel 12 to the steam apparatus 200, and the direct water valve 520 may be constructed to open and close the direct water pipe 510. The direct water pipe 510 may extend from the direct water valve 520 to the steam generator 210 across the support bar 440. In this regard, at least a portion of the direct water pipe 510 may be supported by the support bar 440 to prevent contact with the drying drum 2A.

The support bar 440 may be disposed between the direct water valve 520 and the water tank 430.

In addition, the direct water valve 520 may be seated on the drying rear panel 12 or the second support 19A to be exposed to the outside, and the direct water pipe 510 may extend from the direct water valve 520 toward the steam apparatus 200. Accordingly, the external supply 500 may supply water to the steam apparatus 200 in a direct water scheme from an external water supply source.

The steam apparatus 200 may receive water from each of the external supply 500 and the internal supply 400. However, when the steam apparatus 200 is constructed to receive water through respective pipes, a separate shape of the steam apparatus 200 must be manufactured, so that a flow path and a control method may become complicated.

To this end, the laundry treating apparatus of the present disclosure may further include a combining portion 600 for coupling the direct water pipe 510 with the pump discharge pipe 433. The combining portion 600 may be constructed such that both the water stored in the internal supply 400 and the water supplied in the direct water scheme from the external supply 500 are collected.

In addition, the combining portion 600 may be constructed to deliver the supplied water to the steam apparatus 200. The combining portion 600 may be formed as a three-way valve, or may be formed in a shape of a combining pipe in which three pipes are coupled to each other.

When the combining portion 600 is formed in the pipe shape, a check valve may be disposed in each of the external supply 500 and the internal supply 400 to prevent backflow. Specifically, a direct water check valve 511 for opening the direct water pipe 510 in one direction may be installed on the direct water pipe 510, and the pump discharge pipe 433 may have a discharge check valve 434 for opening the pump discharge pipe 433 in one direction.

In addition, the check valves may be disposed on the pump discharge pipe 433 and the direct water pipe 510, respectively. The external supply 500 may include an external check valve, and the internal supply 400 may include an internal check valve.

Accordingly, the water supplied to the direct water pipe 510 may be prevented from flowing back to the water pump 430, and the water supplied to the pump discharge pipe 433 may be prevented from flowing back to the direct water valve 510.

In one example, the combining portion 600 has a significant weight when formed as the valve or the combining pipe. In addition, when water passes through the combining portion 600, a considerable weight may be applied to the combining portion 600.

Accordingly, the combining portion 600 may be seated on the support bar 440.

The combining portion 600 and the support bar 400 may be coupled with each other using a separate fixing member to prevent the combining portion 600 from being separated from the support bar 400. Because the combining portion 600 is seated on the support bar 400, positions of the direct water pipe 510 and the pump discharge pipe 433 may also be stably fixed.

In one example, the steam apparatus 200 may include a water guide pipe 220 connected to the combining portion 600 to receive water from the water supply 300, a steam generator 210 that receives water from the water guide pipe 220 to generate steam, and a steam guide pipe 230 capable of guiding the steam generated by the steam generator 210 to the drying drum 2A or the duct 3A.

The steam generator 210 may be disposed below the drying drum 2A to stably receive water from the water supply 300 by gravity, and the generated steam may be stably moved to the drying drum 2A by a difference in density.

The steam guide pipe 230 may be constructed to be in communication with a gasket disposed on a front face of the drying drum 2A, or the first support. Accordingly, the steam guide pipe 230 may stably supply steam into the drying drum 2A without being in contact with the drying drum 2A.

In one example, the tank housing 410 may have a mounting sensor capable of sensing whether the water tank 420 is mounted. For example, the mounting sensor may be formed as a pressure sensor or the like.

In addition, a water level sensor capable of sensing a water level of the water tank 420 may be further disposed. For example, the water level sensor may be formed as a weight sensor. The mounted sensor or the water level sensor may also be controlled by the control panel 820, and may be configured to transmit a signal to the control panel 820.

In one example, the control panel P may indirectly identify the water level of the water tank 420 by temporarily driving the water pump 430 to sense a load applied to the water pump 430.

The water storage tank 72 may have a significantly larger volume than the water tank 430, and may be disposed to be spaced apart from the water tank 430 in order to prevent confusion of the user between the water storage tank 72 and the water tank 430.

The water tank 430 and the steam apparatus 200 may be disposed between the support bar 440 and one side face of the cabinet, and the water storage tank 72 may be disposed between the support bar 440 and the other side face of the cabinet.

Because the water tank 430 is coupled to the tank mounting portion 411, it may be seen that the tank mounting portion 411 is also disposed between the support bar 440 and one side face of the cabinet.

Consequently, the support bar 440 may be disposed between the water storage tank 72 and the steam generator 210, and may be disposed between the water storage tank 72 and the water tank 430.

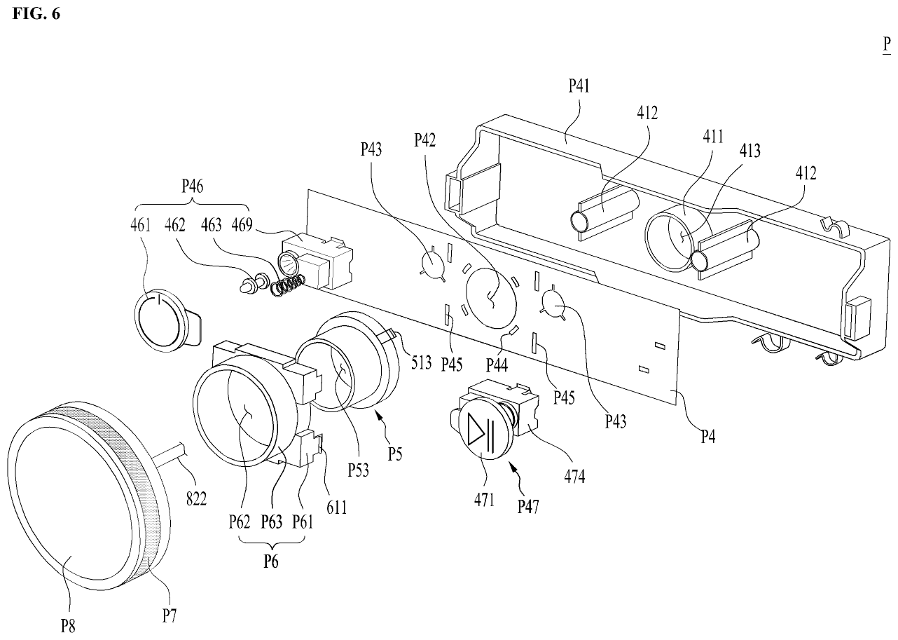

FIG. 6 shows a structure of the control panel P.

The control panel P may include a circuit board (e.g., a first circuit board) P4 fixed to the panel support 12 and positioned inside the cabinet 1, an encoder P5 fixed to the circuit board and located inside the cabinet 1, the manipulation portion P7 connected to the encoder P5 through the front panel 11, and the display portion P8 fixed to the encoder P5 or the first circuit board P4 through the front panel 11.

The first circuit board P4, as a board equipped with a control circuit required for control (e.g., power control and operation control) of at least one of the drivers 23, 27, and 25, a steam apparatus 200, and a water supply 300, may be fixed to the panel support 12 via the casing P41.

The casing P41 may have any shape as long as it is able to fix the first circuit board 4 to the panel support 12.

The casing P41 may be formed in a hexahedral shape with one face (e.g., a face facing the panel support) open.

A boss for setting a position of the first circuit board P4 may be disposed in the casing P41. The boss may include a first boss 411 and a second boss 412.

In this case, the first circuit board P4 may have a board through-hole P42 through which the first boss 411 passes, and a boss insertion hole P43 through which the second boss 412 passes. The second boss 412 may be disposed in each of left and right spaces of the first boss 411, or may be disposed in each of upper and lower spaces of the first boss 411.

A wire 822 is connected to the display portion P8. The wire 822 may be formed as a power line for supplying the power to the displaying interface, or may be formed as a communication line that enables the display portion P8 to communicate with devices inside the cabinet including the first circuit board P4.

The first boss 411 may have a first boss through-hole 413 defined therein, and the panel support 12 may have a wire through-hole 123 (see FIG. 4) defined therein. In this case, the wire 822 may be extended into the cabinet 1 by being inserted into the first boss through-hole 413 and the wire through-hole 123.

The first circuit board P4 may further include the power switch P46 and the executing means P47. The laundry device P46 may be formed as means for inputting the control command for requesting the power supply to the laundry treating apparatus 100, and the executing means P47 may be formed as means for inputting a command for requesting execution of the control command displayed on the display portion P8 or a command for temporarily stopping the control command being executed by the laundry treating apparatus 10.

The power switch P46 and the executing means P47 may be configured to generate a control signal by sensing static electricity of the user's body.

The power switch P46 may include the first button 461 exposed to the outside of the cabinet 1, a first detection sensor 464 fixed to the first circuit board P4, and a conductor (e.g., a first touch spring) 463 for connecting the first button and the first detection sensor to each other. Similarly, the executing means P47 may include the second button 471 exposed to the outside of the cabinet 1, a second detection sensor 474 fixed to the first circuit board P4, and a conductor (e.g., a second touch spring) for connecting the second button and the second detection sensor to each other.

The front panel 11 has the first button mounting portion 136 and the second button mounting portion 117. The first button 461 may be exposed to the outside of the cabinet 1 via the first button mounting portion 116, and the second button 471 may be exposed to the outside of the cabinet 1 via the second button mounting portion 117.

The power switch P46 and the executing means P47 may be separately disposed in the left space and the right space of the display portion P8, may be separately disposed in the upper space and the lower space of the displaying interface, or may be disposed vertically or horizontally in one of the left space and the right space of the displaying interface.

The first touch spring 463 and the second touch spring may be formed in a shape of a coil, which is to provide a restoring force to the first button 461 and the second button 471. Furthermore, in order to prevent the first button 461 and the second button 471 from being respectively deviated from the button mounting portions 116 and 117, the power switch 46 may have a first stopper 462 that limits a range of motion of the first button, and the execution device 47 may have a second stopper that limits a range of motion of the second button.

The encoder P5 is means for rotatably fixing the manipulation portion P7 to the first circuit board P4, and is means for generating an electrical signal (e.g., or generating an electrical signal set differently based on a rotation angle of an actuator) during the rotation of the manipulation portion P7.

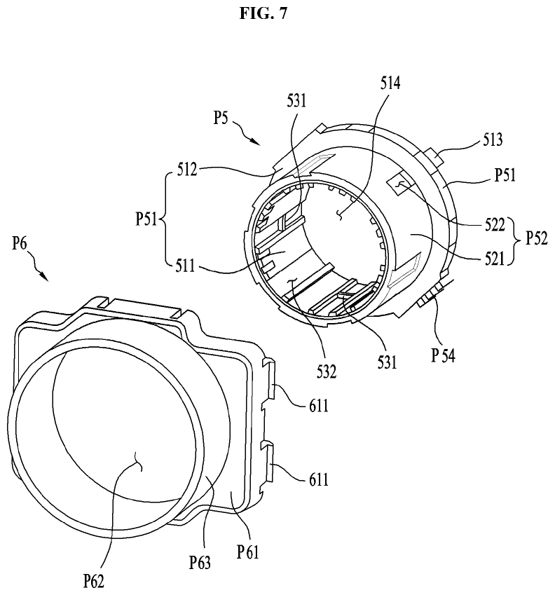

FIG. 7 shows a structure of the encoder.

The encoder P5 may include a fixed portion P51 fixed to the first circuit board P4 and to which the display portion P8 is fixed, a rotating portion P52 rotatably disposed on the fixed portion P51 and to which the manipulation portion P7 is fixed, and a signal generator P54 for generating an electrical signal when the rotating portion P52 is rotated.

The fixed portion P51 may include a fixed body P512 fixed to the first circuit board P4, a support body 511 extending from the fixed body 512 and rotatably fixing the rotating portion P52, and a body through-hole 514 connected to the board through-hole P42 (e.g., connected to the first boss through-hole) through the fixed body and the support body.

The fixed body 512 may be formed in a cylindrical shape. A board fastening portion 513 may be disposed on a circumferential face of the fixed body 512 or on one face (e.g., a face facing the first circuit board) of the fixed body. In this case, the first circuit board P4 may have an encoder fixing hole P44 defined therein into which the board fastening portion 513 is inserted.

The board fastening portion 513 and the encoder fixing hole P44 may respectively include multiple board fastening portions and multiple encoder fixing holes, and the encoder fixing holes P44 may be arranged to surround the board through-hole P42.

Inside the body through-hole 514, a fastening body fixing portion 531 for fastening the display portion P8 to the fixed portion 51, and a position setting groove 532 for preventing the display portion 8 from rotating (e.g., fixing the position of the displaying interface) when the display portion P8 is inserted into the body through-hole 514 may be disposed.

The fastening body fixed portion 531 may be formed as a protrusion protruding toward a center of the body through-hole 514 from at least one of the support body 511 and the fixed body 512. The drawing shows a case in which the fastening body fixed portion 531 is disposed on the support body 511 as an example.

The positioning groove 532 may be defined as a groove in which one face of the fixed portion P51 defining the body through-hole 514 is concavely bent. In this case, the positioning groove 532 may be defined along a longitudinal direction of the body through-hole 514.

The rotating portion P52 may be formed as a rotating body 521 rotatably coupled to the support body 511. The rotating body 521 should have a rotating body through-hole through which the support body 511 passes.

The rotating body 521 may have a shaft fastening portion 522 to which the manipulation portion P7 is fastened. The shaft fastening portion 522 may be formed as a fastening hole extending through a circumferential face of the rotating body 521, or may be formed as a fastening groove defined as the circumferential face of the rotating body 521 is concavely bent.

The signal generator P54 may include a magnet fixed to the rotating body 521, a sensor that is disposed on the fixed body 512 or the support body 511 to sense a magnetic force, and a terminal that connects the sensor and the first circuit board 4 to each other. The magnet may be formed as multiple permanent magnets are arranged to be spaced apart from each other along the circumferential face of the rotating body 521.

The first circuit board P4 to which the encoder P5 is assembled may be coated with an insulating material. This is to minimize a possibility that water is supplied to the first circuit board P4 and the circuit is short-circuited. When the insulating material is coated on one face (e.g., a face facing the front panel) of the first circuit board P4, in order to prevent the rotating body 521 from being fixed to the support body 511 by the insulating material, the control panel P may further include an encoder cover P6.

The encoder cover P6 may be formed in a pipe shape that is fixed to the first circuit board P4 and surrounds the encoder P5. That is, as shown in the drawing, the encoder cover P6 may include a fixed body cover P61 that is fixed to the first circuit board P4 and surrounds the fixed body 512, and a cover through-hole P62 defined to extend through the fixed body cover P61 and into which the encoder 5 is inserted.

The fixed body cover P61 may have a board fastening portion 611, and the first circuit board P4 may have an encoder cover fixing hole P45 to which the board fastening portion 611 is fixed. The board fastening portion 611 and the encoder cover fixing hole P45 may respectively include multiple board fastening portions and multiple encoder cover fixing holes, and the encoder cover fixing holes P45 may be arranged to surround the board fastening portion 513.

For discharging water introduced into the fixed body cover P61, a cover outlet 612 may be further disposed on a circumferential face of the fixed body cover P61. The cover outlet 612 is preferably located at a lowermost end of the circumferential face of the fixed body cover P61.

In one example, the above-described insulating material is applied to the first circuit board P4 after both the encoder P5 and the encoder cover P6 are coupled to the first circuit board P4, and the insulating material sprayed on a partial area of the first circuit board P4 (e.g., an outer space of the encoder cover) flows into the encoder cover P6 through the cover outlet 612. In such a process, in order to block the insulating material from being supplied to the rotating body 521, a distance L1 from the first circuit board to an uppermost end of the fixed body 512 is preferably set to be equal to or greater than a distance L2 from the first circuit board to an uppermost end of the cover outlet 612.

The encoder cover P6 may further include a support body cover P63 extending from the fixed body cover P61 and surrounding the rotating body 521 (e.g., surrounding the support body). The support body cover P63 may limit the movement of the manipulation portion P7 in a radial direction of the cover through-hole 62, so that the support body cover 63 may prevent the manipulation portion P7 from being separated from the rotating body 521.

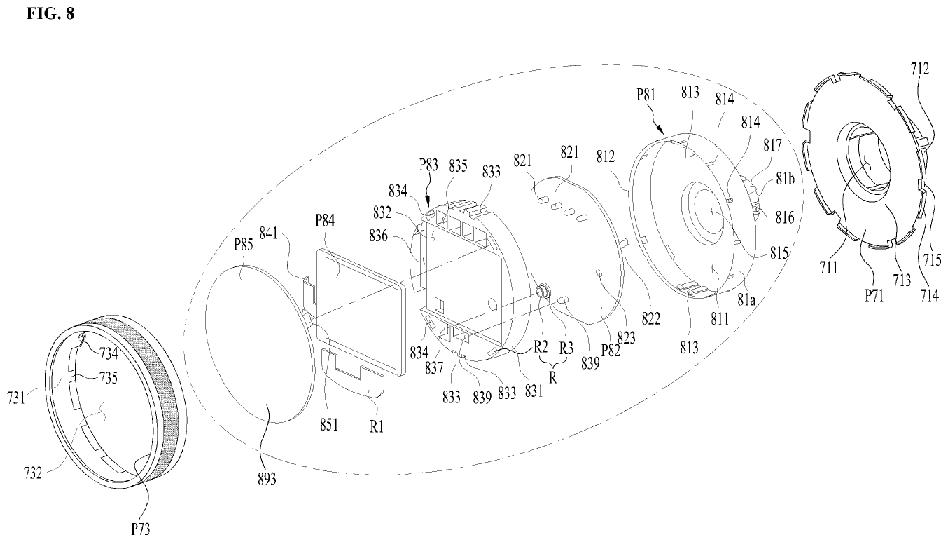

FIG. 8 shows a structure of the manipulation portion P7 and the display portion P8.

The manipulation portion P7 may include a shaft P71 that is fixed to the rotating body 521 through the front panel 11, and a handle P73 fixed to the shaft P71 and positioned outside the cabinet 1.

The cabinet 1 has the panel through-hole 134 defined to extend through the front panel 11. The shaft P71 may be disposed to be inserted into the panel through-hole 134.

The shaft P71 may be formed a pipe having a shaft through-hole 711 defined therein. The rotating body 521 disposed in the encoder is inserted into the shaft through-hole 711, and a rotating body fastening portion 712 disposed on a circumferential face of the shaft P71 and positioned inside the shaft through-hole 711 is coupled to a shaft fastening portion 522 disposed on the rotating body. Accordingly, the shaft P71 is fixed to the rotating body 521 via the rotating body fastening portion 712 and the shaft fastening portion 522.

The handle P73 may be fixed to the shaft P71 so as to be located outside of the cabinet 1, and the user may provide a force required for the rotation of the rotating body 521 to the shaft P71 via the handle P73.

The handle P73 may include an accommodation space 731 defined inside the handle to be in communication with the shaft through-hole 711, and a handle through-hole 732 that extends through one face of the handle P73 to allow the accommodation space 731 to be in communication with the outside.

When a diameter of the handle P73 is set to be larger than a diameter of the shaft P71, the shaft P71 may further include a base 713 to which the handle P73 is fixed. The base 713 may be formed as a disk fixed to the circumferential face of the shaft P71.

The handle P73 and the base 713 may be coupled to each other via a base fastening portion 734 disposed on the handle 73 and a handle fastening portion 714 disposed on the base 713.

The base fastening portion 734 may be formed as multiple protrusions fixed to the handle so as to be located inside the accommodation space 731, and the handle fastening portion 714 may be formed as a groove defined in the base 713 and accommodating the protrusion therein.

In order to facilitate the coupling of the handle P73 and the base 713, the handle P73 may have a fastening guide 735 positioned between one base fastening portion 734 and another base fastening portion 734, and the base 713 may have a guide groove 715 into which the fastening guide 735 is inserted. The guide groove 715 may be defined as a groove in which a circumferential face of the base 713 is bent toward the shaft through-hole 711.

The display portion P8 may include a housing P81 fixed to the fixed body 512 of the encoder and located inside the accommodation space 731 of the actuator, the display P84 fixed to the housing and displaying information (e.g., information related to control, operation, and the like of the laundry treating apparatus), and a circuit board (e.g., second circuit board and a display portion circuit board) P82 disposed in the housing and having a circuit for controlling the display P84.

The housing P81 may include a fastening body 81b fixed to the fixed body 512 by extending through the shaft through-hole 711 and being inserted into the body through-hole 514, and an accommodating body 81a fixed to the fastening body 81b and positioned inside the accommodation space 731.

The accommodating body 81a may be formed in any shape as long as it may be inserted into the accommodation space 731. A case in which the accommodating body 81a is formed in a cylindrical shape is shown as an example.

A mounting space 811 is defined inside the accommodating body 81a, and the mounting space 811 is in communication with the accommodation space 731 defined in the handle via an accommodating body through-hole 812 (e.g., the accommodating body through-hole is defined to be in communication with the handle through-hole). That is, the accommodating body through-hole 812 is defined in a face directed in a direction in which the handle through-hole 732 is located of the space provided by the accommodating body 81a.

The fastening body 81b may be formed in any shape as long as it is able to be inserted into the shaft through-hole 711 and the body through-hole 514 of the encoder. A case in which the fastening body 81b is formed in a cylindrical shape is shown as an example. The fastening body 81b has a fastening body through-hole 815 connected to the mounting space 811.

A fixed body fastening portion 816 may be disposed on a circumferential face of the fastening body 81b. The fixed body fastening portion 816 is means for fixing the housing P81 to the fastening body fixing portion 531 disposed in the encoder.

Further, multiple position setting protrusions 817 may be further disposed on the circumferential face of the fastening body 81b.

The position setting protrusion 817 is formed in a shape capable of being inserted into the position setting groove 532 defined in the encoder, and the position setting protrusion 817 is disposed at a point corresponding to a position of the position setting groove 532. The position setting protrusion 817 and the position setting groove 532 are means for minimizing a risk that the fixed body fastening portion 816 fails to be coupled to the portion 531 (e.g., means for ensuring the coupling of the fixed body fastening portion and the fastening body fixing portion) when the fastening body 81b is inserted into the body through-hole 514.

The second circuit board P82 may be inserted into the mounting space 811, and the wire 822 connected to the second circuit board P82 may be withdrawn to the outside of the housing P81 through the fastening body through-hole 815. Multiple lamps 821 may be disposed on the second circuit board P82.

The second circuit board P82 may control the display P84 to display preset information in response to an electrical signal generated from the signal generator 54 when the handle P73 is rotated.

In order to maintain a gap between the second circuit board P82 and the display P84 and to prevent damage to the lamp 821, a mounting portion P83 positioned between the display P84 and the second circuit board P82 may be disposed in the mounting space 811.

The mounting portion P83 may be formed as a mounting body 831 fixed to the accommodating body 81a and positioned inside the mounting space 811. The mounting body 831 may have any shape as long as it is able to be inserted into the mounting space 811.

A seating groove 832 to which the display P84 is fixed is defined in one face of the mounting body 831. The seating groove 832 may have a connector through-hole 836. A connector 841 (e.g., a flexible PCB or the like) disposed in the display P84 may be connected to the second circuit board P82 by being inserted into the connector through-hole 836.

The mounting body 831 may have a lamp through-hole 835 into which the lamp 821 is inserted. It is preferable that the number of lamp through-holes 835 is the same number as the number of the lamps 821, and a position of the lamp through-hole 835 should be set to a space of the mounting body located above the mounting groove 832 or a space of the mounting body located below the mounting groove 832. This is for light emitted from the lamp 821 to be transmitted to the outside of the housing P81 through the lamp through-hole 835.

The mounting portion P83 is fixed to the housing P81 via a mounting body fastening portion 813 disposed on the accommodating body 81a and an accommodating body fastening portion 833 disposed on the mounting body 831. The mounting body fastening portion 813 may be formed as multiple protrusions located inside the mounting space 811, and the accommodating body fastening portion 833 may be defined as a groove or the like defined in the circumferential face of the mounting body 831 (e.g., one face of the mounting body in contact with the accommodating body) and to which the mounting body fastening portion 813 is fixed.

The number of mounting body fastening portions 813 and the number of accommodating body fastening portions 833 should be set to be the same, and positions of the mounting body fastening portion 813 and the accommodating body fastening portion 833 should be set to correspond to each other.

In order to increase a fastening force of the mounting body 831, the accommodating body fastening portion 833 may include an upper fastening portion positioned higher than the seating surface 832 and a lower fastening portion positioned lower than the seating surface 832. Unlike the drawing, the accommodating body fastening portion 833 may be disposed on each of left and right sides of the seating face.

A cover P85 may be further disposed on the accommodating body 81a to prevent water and foreign substances from flowing into the display P84 and the second circuit board P82.

Preferably, the cover P85 is fixed to at least one of the accommodating body 81a and the mounting body 831 to close the accommodating body through-hole 812. The cover P85 should be made of a material having a transparency that allows the information displayed on the display P84 and the light emitted from the lamp 821 to be viewed from the outside.

When the cover P85 is fixed to the mounting body 831, the cover P85 may have a cover fixing protrusion 851 protruding toward the mounting body 831, and the mounting body 831 may have a fixing protrusion fastening portion 834 to which the cover fixing protrusion 851 is coupled.

The second circuit board P82 may also be fixed to the mounting body 831. That is, the mounting portion P83 may have a protrusion 838 protruding from the mounting body 831 toward the second circuit board 82, and the second circuit board 82 may have a protrusion through-hole 823 into which the protrusion 838 is inserted.

Further, the cover P85 may further include a cover sheet 853 for lowering the transparency of the cover. It is preferable that a transparency of the cover sheet is set to such a degree that it is difficult to view the interior of the mounting space 811 from the outside (e.g., the transparency of the cover sheet is set lower than the transparency of the cover). The cover sheet 853 may be fixed to a surface of the cover P85, and a letter or symbol H may be displayed in an area of the cover sheet on which the lamp through-hole 835 is projected.

In one example, a lamp through-hole 835 corresponding to the input area D3 among the lamp through-holes 835 in the mounting portion P83 may provide a signal instructing the user to press the input area D3.

The selection portion R that receives the control command of selecting the control command displayed on the display 84 may be further disposed on the control panel P.

The selection portion R may be configured to determine whether the control command displayed on the display P84 is selected by sensing whether the user's body is in contact with the cover P85.

That is, the selection portion R may include a sensor R3 disposed on the second circuit board 82 and sensing the static electricity of the user's body, and a conductor R2 or a physical button R1 for connecting the sensor R3 and the window P85 to each other.

The selection portion R may be composed of a disposed portion R1 attached to or coupled to the cover P85, and a transmitter R2 having one end connected to the disposed portion R1 and the other end connected to the sensor R3. In this case, the mounting body 831 must have a conductor through-hole 837 into which the transmitter R2 is inserted.

The disposed portion R1 is means for facilitating the input of the control command by expanding an area that the user may contact. In the control panel P having the above structure, the display area D1 is formed in an upper space of the cover P85, and the input area D3 is formed in a lower space of the cover P85. Therefore, when a width of the disposed portion R1 is set to be great, a width of the input area D3 is expanded, so that the user may more easily input the control command. The width of the disposed portion R1 is preferably set to equal to or smaller than ¼ to ½ of a width of the cover P85.

The transmitter R2 and the disposed portion R1 may be formed as a conductor. Therefore, when the user's body is in contact with the transmitter R2 and the receiver R1, a current of the user's body may be charged or transmitted, so that the current may be transmitted to the sensor R3.

In addition, the transmitter R2 and the disposed portion R1 may be formed physical buttons. Therefore, when the user's body presses the disposed portion R1, the pressure of the body may be transmitted to the sensor R3.

In one example, the display portion P may include inducing means 839 for inducing the user to press the selection portion R.

The inducing means 839 may guide the user to the location of the selection portion R to induce the user to press the selection portion R.

The inducing means 839 may include the lamp 821 and the lamp through-hole 835 corresponding to the position of the disposed portion R1. Specifically, the inducing means 839 may include the lamp through-hole 835 defined below the display P84 and the lamp 821 installed inside the lamp through-hole 835.

The lamp through-hole 835 and the lamp 821 constituting the inducting means 839 may be disposed below the display P84 and at a center of the mounting portion 83.

Accordingly, when the lamp 821 emits the light in the inducting means 839, the selection portion R or an area adjacent to the selection portion R may be brightened.

In addition, the lamp 821 of the inducing means 830 may be formed as an LED element and the like to indicate a separate character or phrase.

The control panel P having the above structure enables display of the control command, search for the displayed control command, and selection of the displayed control command, and enables minimization of a space required for installation. However, the control panel P having the above structure needs means to block the inflow of water or the foreign substances to the display portion P8 and the manipulation portion P7 or to discharge the inflowed water or foreign substances to the outside.

The control panel P having the above structure will display a preset control command on the display P84 whenever the user rotates the handle P73 (e.g., whenever the encoder generates the electrical signal) (e.g., search and change the control command).

When the desired control command is displayed on the display P84, the user may select the displayed control command via the selection portion R, and the control command selected via the selection portion R may be initiated when the user inputs an execution command via the executing means P47.

In one example, the handle P73 is coupled to the first circuit board P4 to enable a rotational movement, whereas the housing P81 of the display portion is fixed to the first circuit board P4, so that it is necessary to keep a gap between the handle P73 and the housing P81 constant. This is because, when the gap between the handle P73 and the housing P81 is not maintained, the rotation of the handle 7P3 is likely to be constrained.

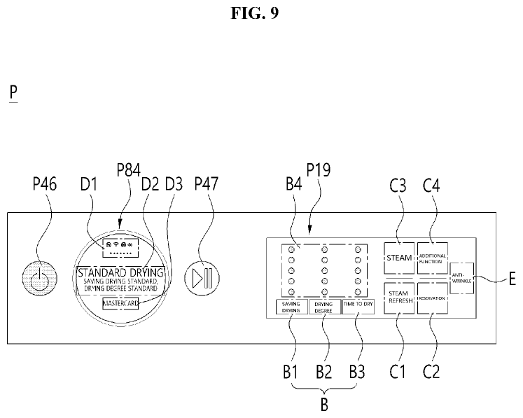

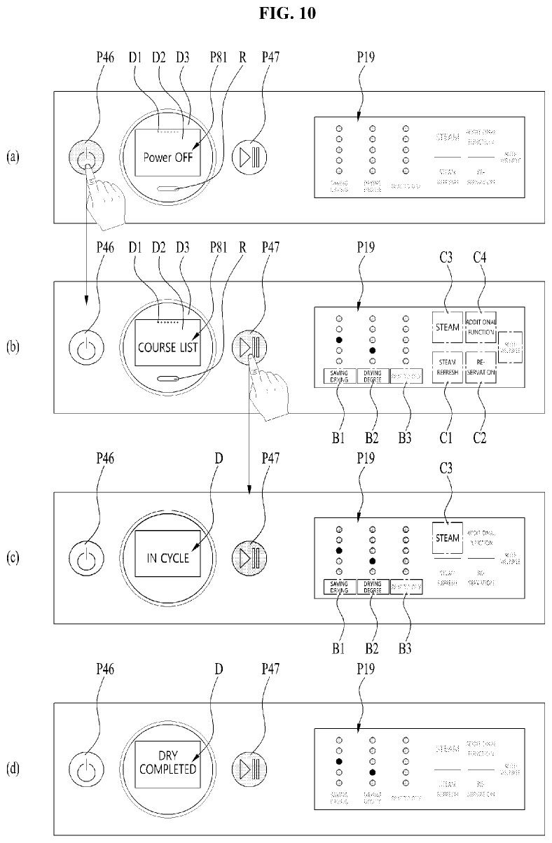

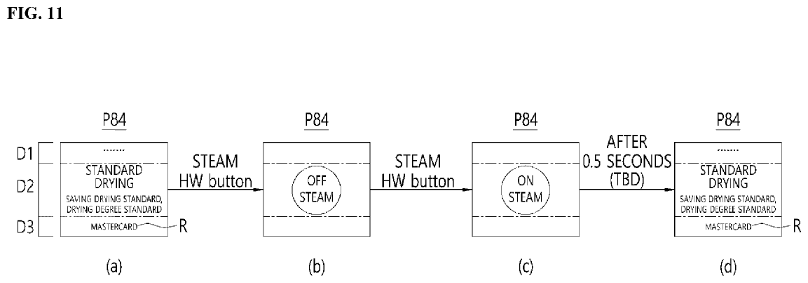

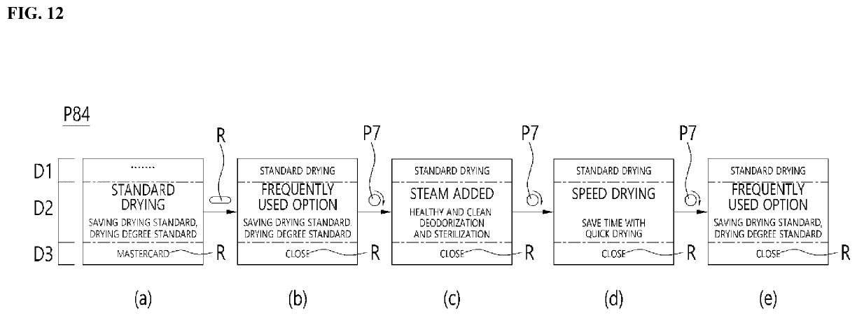

FIG. 9 shows an embodiment of utilization of the control panel P of the laundry treating apparatus of the present disclosure.

In the state display area D1 of the display portion P8, whether the door is locked, whether the communication module is activated, whether the course or the option is operated, whether the guide phrase is generated, and the like may appear in a form of an icon.

In addition, the number of displayed contents that may appear while rotating the manipulation portion P7 may be displayed in the state display area D1. For example, a plurality of dots may be arranged to be spaced apart from each other, and the number of contents displayed in the content display area D2 may be provided while one of the plurality of dots emits the light.

In the content display area D2, contents of the course and the option, the executed state of the course or the option, and the guide phrase necessary for the user may be displayed.

The content display area D2 may be larger than the state display area D1.

In the input area D3, light may be emitted to request the input of the user, and the guide phrase indicating which content the user should input may be displayed.

The power switch P46 may be disposed at a left side of the display portion P8, and the executing means P47 may be disposed at a right side of the display portion P8.

The manipulation portion P7 may be rotatably disposed on an outer circumferential face of the display portion P8, and may be configured to select an arbitrary course capable of rotating the drum 2 or operating the steam apparatus 200.

The arbitrary course may be a series of control methods for performing the drying cycle of drying the laundry.

For example, the arbitrary course may include a standard course for drying a general standard laundry, a wool course for drying laundry with a high moisture content and a high risk of shrinkage, such as wool, and a steam course for removing wrinkles from or sterilizing the laundry by spraying steam on the laundry/

When the manipulation portion P7 is rotated, a name of the corresponding course and a simple option name may be displayed in the content display area D2 of the display portion P8 based on a set order, and a description of the course or the option may be displayed.

The option may be selected by the setting portion P2. The setting portion P2 may be configured to receive a selection command for selecting at least one of the arbitrary options.

The arbitrary option may include setting additional conditions of the course.

For example, the arbitrary option may be a set algorithm or a control method for adjusting at least one of the number of repetitions of each cycle to perform the course, an intensity of performing each cycle, a rotation speed of the drum when performing each cycle, a temperature of water, an amount of water, and a duration of each cycle.

The setting portion P2 may be formed as a touch display for selecting the arbitrary option, and may have a plurality of lamps and a plurality of conductor switches capable of sensing the user's body.

For example, the setting portion P2 may include option selection portion B that receives a setting command to set the conditions for the course, and option display portion B4 that displays option information corresponding to the option selection portion B.

For example, the option selection portion B may include a saving drying interface B1 for determining a degree of electric energy applied when executing the arbitrary course, a dryness selection portion B2 for determining a degree of the moisture content or a drying rate of the laundry when the drying cycle of the laundry is terminated, and a time drying interface B3 for determining a duration of the course.

The option display portion B4 may be constructed to display electric energy, a drying rate, and a duration of a corresponding course in a numerical and unitized manner, and may be constructed to also display a degree of change.

In one example, the setting portion P2 may have utilizing means C that receives a utilization command to utilize a function of the laundry treating apparatus.

For example, the utilizing means C may include a steam interface C3 for spraying steam when performing the arbitrary course, refresh means C1 for performing steam refresh that removes odors and volatile substances from the laundry by supplying hot air and steam to the laundry, reservation means C2 for inputting a reservation command to perform the course at a specific time, and additional function means C4 for utilizing additional functions such as pairing with other devices.

In addition, the setting portion P2 may further include an anti-wrinkle interface E for executing an anti-wrinkle course for preventing the wrinkling of the laundry when the course is terminated.

The setting portion P2 may allow a function that may be utilized at a present time point or a function selected by the user to be turned on and allow a function that is not able to be executed or is not selected by the user to be turned off.

The setting portion P2 may display all of the contents via an auxiliary display P19.

FIG. 10 shows an aspect of the control panel P when operating the laundry treating apparatus of the present disclosure.

Referring to (a) in FIG. 10, the laundry treating apparatus is in a state in which the power supply is cut off so that the display portion P8 and the auxiliary display P19 are turned off.

The user may supply power to the laundry treating apparatus including the display portion P8 and the auxiliary display P19 by inputting the power command to the power switch P46.

Referring to (b) in FIG. 10, the power may be supplied to the control panel P and the manipulation portion P7 may be activated. The user may select the desired course by rotating the manipulation portion P7 and may select the desired option by checking or pressing the auxiliary display P19.

The selected course and option may be displayed on the display portion P8, and a selected additional option or function may be displayed on the auxiliary display P19.

The user may execute the course and the option by pressing the executing means P47.

Referring to (c) in FIG. 10, the display portion P8 may display the executed state of the course and the option, and the auxiliary display P19 may display a current state of the option. For example, when the additional function such as the steam is used, the steam interface C3 may be turned on. In addition, when the drying course is performed with a medium amount of energy of the course and a low dryness, the fact that the moisture content is high and a drying time is not particularly limited may be displayed.

Referring to (d) in FIG. 10, completion information indicating that execution of the course and the option has been completed may be displayed on the display portion P8. Thereafter, the power supply to the control panel P may be cut off.

The user may determine the course or the option from the selection of the course or the option based on the information displayed on the display portion P8, and the executed state of the course or the option and the completed state of the course or the option may be identified at once.

FIG. 11 shows utilization of the display portion P8, which emphasizes a portion of the display P84 of the display portion P8.

Referring to (a) in FIG. 11, when power is input to the control panel P, a name and a description of the selected course or option may be described in the content display area D2 of the display portion P8 or the display P84, and information on the number of courses or options that may be changed may be displayed in the state display area D1.

The content display area D2 may be disposed beneath the state display area D1, and may have a larger area than the state display area D1. Accordingly, the content display area D2 may actively transmit information necessary to the user using characters, icons, and the like.

The state display portion D1 may intuitively transmit the information via a symbol such as an icon or a sign.

The input area D3 may be disposed beneath the content display area D2. Accordingly, when the user touches the input area D3, the user's body may be prevented from covering the content display area D2.

The input area D3 may display information that allows the user to determine the information displayed in the content display area D2. That is, when it is necessary to receive a command that requires confirmation or determination of the user via the selection portion R, input contents corresponding to the selection portion R may be displayed in the input area D3.

For example, rather than the arbitrary course or option set as a default that may be selected by rotating the manipulation portion P7, an indication that a course or option in a form of a MasterCard additionally stored in a controller such as a recommended course or option provided by a producer or the like, a course or option that has been used N times or more recently, a course or option preset by the user, or the like may be displayed in the input area D3.

The MasterCard, which corresponds new courses or options provided from the server or set by the user via changing an option value for a specific course, may correspond to courses or options that are different from the arbitrary course or option set as the default when the laundry treating apparatus is purchased.

Referring to (b) to (c) in FIG. 11, the display P84 may display a confirmation content for confirming the command additionally input by the user. The confirmation content may be a name or an effect of the command input by the user that is displayed in an enlarged manner.

For example, when the user presses the steam interface C3 of the setting portion P2, not only the steam interface C3 is turned on the auxiliary display P19, but also a phrase such as steam-on appears in a large size on the display P84 to indicate to the user that the steam function is activated.

In addition, when the user re-presses the steam interface C3 of the setting portion P2, not only the steam interface C3 is turned off on the auxiliary display P19, but also a phrase such as steam-off appears on the display P84 to indicate to the user that the steam function is deactivated.

Accordingly, the user may intuitively identify what the command he or she has inputted and whether the command thereof has been properly input via one display P84. Accordingly, even when there is no separate display device for separately displaying whether the corresponding command has been received, the user may accurately determine whether the corresponding command has been input.

Referring to (d) in FIG. 11, after waiting for a certain period of time or when the selection portion R is pressed, the selected course and option may be displayed on the display P84. Accordingly, the user may identify that the command thereof has been reflected, and may execute the selected course and option without delay in the course execution.

FIG. 12 shows an embodiment of selecting a course or an option.

In the control panel P, the arbitrary course or option that is provided by default may be stored. However, the user may continuously add a specific option in a specific course and repeatedly use the added option.

In addition, there may be a course or an option that may be recommended by the producer to the user when researching a weather, an installed area, characteristics of the laundry treating apparatus, and the like.

However, it may be very cumbersome for the user to repeatedly set such course or option, and the user may not be able to use the corresponding course or option because the user is not aware of the corresponding course or option.

Accordingly, the control panel P of the laundry treating apparatus of the present disclosure may separately store a new course or option rather than the course or the option set as the default in a form of a shortcut.

For example, the newly set courses or options may be sequentially displayed on the display P84 by the input of the command to the manipulation portion P7. Accordingly, because types of new courses or options are sequentially displayed on the display portion P84, this may be defined as the MasterCard.

That is, the MasterCard may be one of the newly set courses and options, rather than the courses and options provided by default. There may be plurality of MasterCards and an order in which the plurality of MasterCards are sequentially displayed may be set in advance.

For example, a MasterCard corresponding to a course frequently used by the user or a course set in advance by the user may be displayed first, followed by a recommended course.

Referring to (a) in FIG. 12, a standard drying course, which is one of the courses or the options provided by default may be displayed on the display P84.

In this regard, the user may select the MasterCard displayed in the input area D3 via the selection portion R.

Referring to (b) in FIG. 12, a first MasterCard may be displayed on the display P84. The first MasterCard may be an option frequently used by the user recently.

The user may execute a course corresponding to the MasterCard by inputting the command to the executing means. However, the user may select another MasterCard by rotating the manipulation portion P7.

Referring to (c) in FIG. 12, when the user rotates the manipulation portion P7, a second MasterCard may appear. For example, when the laundry treating apparatus has the steam function, an option in which the steam is added may be recommended.

Referring to (d) in FIG. 12, when the user rotates the manipulation portion P7 again, a third MasterCard may appear. For example, a speed drying course capable of maximally reducing a drying time compared to a general drying course may appear. This is to increase a temperature or a flow rate of hot air, which may be applied when a material of laundry has a great durability.

Referring to (e) in FIG. 12, when the user rotates the manipulation portion P7 again, a fourth MasterCard may appear, which may be an option frequently used by the user recently.

As such, the user may identify the stored MasterCards sequentially by rotating the manipulation portion P7. The first MasterCard may reappear when every MasterCard is identified.

Accordingly, even when input means for inputting a separate course or option is not provided, the laundry treating apparatus of the present disclosure may rapidly select the desired course and option via the manipulation portion P7.

As described above, the laundry treating apparatus of the present disclosure may automatically wash the heat exchanger assembly 4 via the washer 6. However, the user may not even know that the laundry treating apparatus of the present disclosure has a function to automatically wash the heat exchanger assembly 4, may not know what to wash the heat exchanger assembly 4 with, and may misunderstand that there is an abnormality in the laundry treating apparatus because of not knowing what phenomenon occurs when washing the heat exchanger assembly 4.

In order to prevent the above problems, the laundry treating apparatus of the present disclosure may display operation information of the pump on the display portion P8 when a condenser is washed.

The operation information D2A of the pump may include one of a wash phrase D21 indicating a state in which the heat exchanger is washed by water, an operation phrase D22 indicating a state in which the pump is operating itself, and a noise phrase D23 indicating a state in which noise may be generated in the pump.

As such, the user may understand that the laundry treating apparatus of the present disclosure has the function of automatically washing the condenser when executing the drying course and option while identifying the operation information D2A displayed on the display P84.

Specifically, the laundry treating apparatus of the present disclosure may display the wash phrase D21 via the display portion P8, so that the user may understand that the condenser is automatically washed with water, and may advertise that the laundry treating apparatus is clean as the foreign substances and the lint are automatically removed therefrom.

In addition, the laundry treating apparatus of the present disclosure may display the operation phrase D22 via the display portion P8 to notify the user that the separate pump is installed to clean the condenser, and may advertise that the laundry treating apparatus has excellent cost-effectiveness.

In addition, the laundry treating apparatus of the present disclosure may display the noise phrase D23 via the display portion P8 to transmit information indicating that the pump generates the noise while being operated, and may reassure the user that there is no problem with the laundry treating apparatus.

The display portion P8 may display one of the wash phrase, the operation phrase, and the noise phrase.

In addition, the display portion P8 may alternately display at least two of the wash phrase, the operation phrase, and the noise phrase.

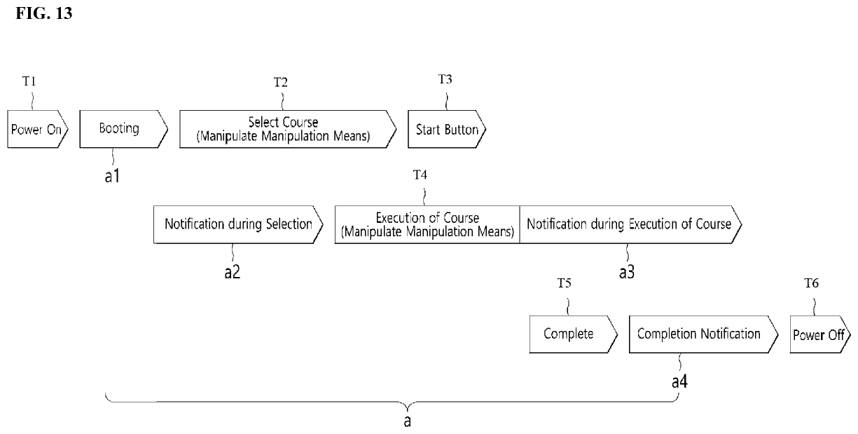

FIG. 13 shows a notification manner according to an operation process of a laundry treating apparatus according to the present disclosure.

The laundry treating apparatus according to the present disclosure may perform a course and an option through the following process.

The laundry treating apparatus according to the present disclosure may perform a power supply step T1 of supplying power to one or more of the control panel P and the driver 28 when receiving input of the power switch P47.

When power is supplied to the control panel P, the manipulation portion P7 and the display portion P8 may be activated, and a course selection step T2 in which a user selects a desired course and option by manipulating the manipulation portion P7 and a setting portion P2 may be performed.

An execution step T3 of executing the selected course or option when receiving input of the executing portion P47 may be performed.

When the execution step T3 is performed, a drying step T4 in which laundry is dried by operating the driver 28, a heat exchanger 4, a washer 6, the steam part 200, and the water supply 300 may be performed according to a control method determined by the course and the option.

When the drying step T4 is completed, a cooling step T5 of lowering a temperature inside the drum 2 may be performed. The cooling step T5 may be a step in which the heat exchanger 4 is not driven and only a blower fan is driven or a step in which the laundry treating apparatus is on standby for a predetermined time until the temperature drops below a safe temperature. The safe temperature may be set below 30 degrees.

When the cooling step T5 is terminated, a power-off step T6 in which the course and the option are completed and power is shut off may be performed.

When the laundry treating apparatus is operated to perform the course and the option, the laundry treating apparatus may provide notification information A required for a user.

For example, the laundry treating apparatus according to the present disclosure may provide the notification information A at a booting time point a1 of performing the power supply step T1 and may provide the notification information A at a time point of selecting a course in the course selection step T2 or a selection time point a2 at which the corresponding course and option are selected to be executed in the execution step T3.

The laundry treating apparatus according to the present disclosure may provide notification information at an execution time point a3 when executing the course and the option in the drying step T4 and may provide the notification information at a completion time point a4 that corresponds to the end of the cooling step T5 and just before the power-off step T6.

The laundry treating apparatus according to the present disclosure may further include a steam course for spraying steam while drying laundry.

In the steam course, the steam generated in the steam part 200 may be sprayed to laundry dried with the hot air.

The steam part 200 may receive water from the water supply 300 and may heat the water to a boiling point to generate steam. Since the temperature of the steam corresponds to more than 60 degrees Celsius at which bacteria die, when the steam is sprayed on the laundry, the bacteria may be removed.

When steam is sprayed to laundry with reduced moisture content as drying progresses considerably, wrinkles may be removed from the laundry, and the laundry may be prevented from being contracted by high heat.

Since the steam is evaporated while being attached to foreign substances attached to the laundry, the laundry may also be refreshed.

The steam course may be a concept including all courses for spraying steam to the laundry, and may be added while an arbitrary course and option and may be executed together. However, in order to perform the steam course, water for generating steam needs to be supplied to the steam part 200.

However, in the water supply 300, the external supply 500 may not be connected to an external water supply source, or the internal supply 400 may not be filled with enough water sometimes.

In this case, even if the steam course is executed, steam may not be sprayed. Even if steam is not sprayed during selection of the steam course, the course and the option may be executed without change in order to drying delay.

As a result, when water is not supplied to the steam part 200, the user may not experience an effect of the steam course irrespective of whether the steam course proceeds. The user may doubt steam performance of the laundry treating apparatus.

In order to prevent this, when water supply to the steam part 200 is insufficient, the laundry treating apparatus according to the present disclosure may be configured to display this as the notification information A. Thus, when the steam course is used, steam may be induced to be normally sprayed.

The control panel P may be configured to detect a state in which water supply is insufficient to the steam part 200. The control panel P may detect whether water is insufficient through a water level sensor mounted inside the steam part 200 and may detect whether water is insufficient by detecting a water level of the water tank 430 or whether the direct water valve 520 is opened or closed.

The insufficient water supply may include that a water level of the steam part 200 is lower than an appropriate amount of water required for the steam part 200 to perform the steam course.

When water supply to the steam part 200 is insufficient, the control panel P may generate insufficiency information and may display the insufficiency information through the display portion P8. The insufficiency information may use any text, icon, and symbol as long as it is possible to indicate that the steam part 200 is not capable of generating steam to the user.

When the insufficiency information is generated, the display portion may be controlled to display the insufficiency information at at least one of the booting time point a1 at which power is supplied to the display portion, the selection time point a2 of a course or an option of input of a manipulation portion, the execution time point a3 at which the course or the option is executed, or the completion time point a4 at which the course or the option is completed.

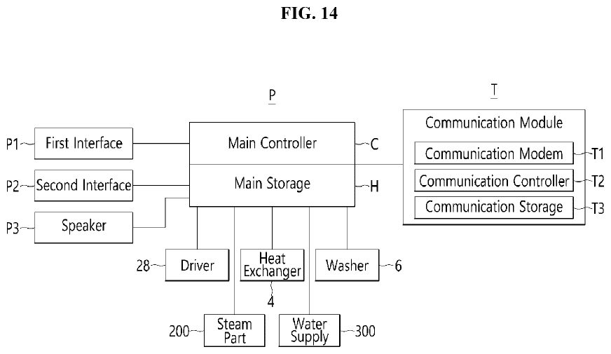

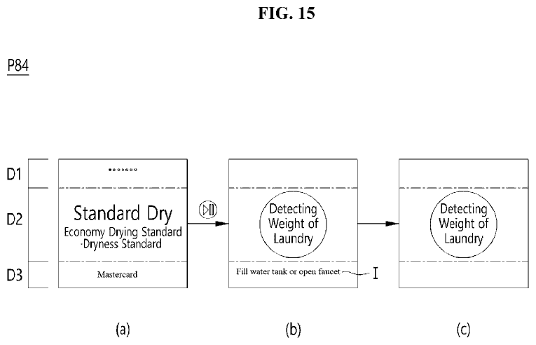

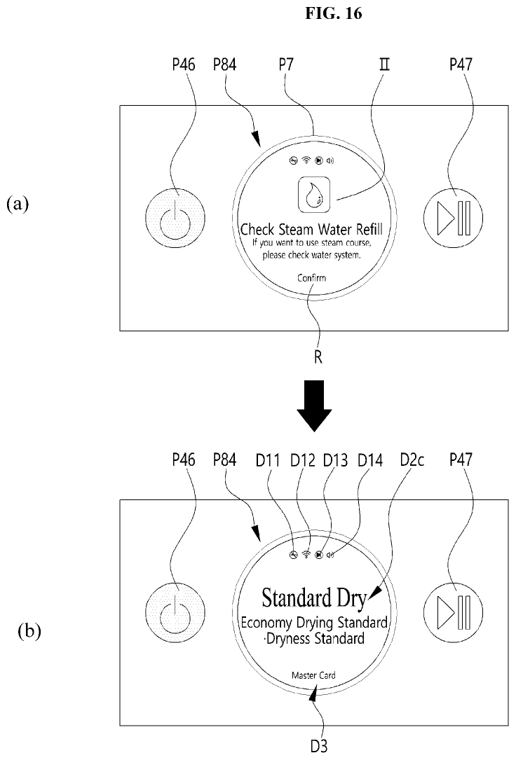

FIG. 14 shows a control block diagram of a laundry treating apparatus according to the present disclosure.

The laundry treating apparatus according to the present disclosure may include a main controller C that controls the driver 28, the heat exchanger 4, the washer 6, the steam part 200, and the water supply 300 to execute a course and an option.

The main controller C may be installed on a separate circuit board accommodated inside the cabinet 1, and may be installed on a circuit board P4 accommodated on the control panel P. In this case, the control panel P and the main controller C may also be considered as functionally identical components.

The main controller C may be provided to control a case P1 or a first interface p1 on which the manipulation portion P7, the display portion P8, the power switch P46, and the executing portion P47 are installed.

The main controller C may be configured to control the setting portion P2 or a second interface p2.

The main controller C may be configured to control a separate speaker p3 coupled to the cabinet 1.

The control panel P may include a main storage H for storing a program and an algorithm for operating the main controller C. The main storage H may download and store external information from a communication module T to be described later and may provide the external information to the main controller C.

The laundry treating apparatus according to the present disclosure may be provided to communicate with an electronic product disposed outside the cabinet 1 through the communication module T.

The communication module T may include a communication modem T1 configured to communicate with the electronic product, a communication controller T2 for controlling the communication modem, and a communication storage T3 configured to store a program for controlling the communication controller T2 or information received through the communication modem.

The main controller C may control the steam part 200 and the water supply 300 to detect a state in which water is insufficient in the steam part 200 and may generate the insufficiency information. The insufficiency information may be stored in the main storage H and may be provided as notification to the display portion P8 when power is supplied to the control panel P in the future.

Thus, the laundry treating apparatus according to the present disclosure may store the insufficiency information in the storage when generating the insufficiency information, and the display portion P8 may display the insufficiency information at the booting time point a1 when the main controller C detects that the insufficiency information is present in the storage at the booting time point a1.

Accordingly, the user may arrange to supply water to the steam part 200 before selecting the course and option. In addition, the user may choose not to execute a steam course or may select the course and the option without expecting a steam effect.

Whether to provide steam may not be required to dry laundry. Therefore, even in a situation in which steam is not capable of being supplied, when the laundry treating apparatus is on standby for a long time, there is a risk that wet laundry is corrupted or excessive wrinkling occurs. In addition, drying time may be delayed.

To prevent this, when the insufficiency information is displayed on the display portion P8 for a reference time, the display portion P8 may remove display of the insufficiency information and may return to an original state.

For example, when the insufficiency information is displayed at the booting time point a1 on the display portion P8 for a reference time, display of the insufficiency information may be removed. Thus, the user may recognize that water supply is insufficient in the steam part 200, and may select any course and option regardless of this.

The reference time may be set to a booting time or a time between 1 second and 10 seconds.

FIG. 15 show an embodiment in which the insufficiency information is displayed.

The insufficiency information may be displayed on the display portion P8 in a pop-up form I. Thus, even if water is insufficient in the steam part 200, it may be possible to prevent delay of the course and the option.

In addition, the user may recognize a situation in which water is not sufficiently supplied to the steam part 200 through notification of insufficiency information in the pop-up form I. In other words, the user may check the insufficiency information, but may also check a state in which the course and option are selected or executed at the same.

For example, referring to (a) of FIG. 15, when the booting time elapses, a selection step T2 may be performed on the display portion P8 to display the selected course and option.

The number of courses and options to be selected may be displayed in the state display area D1, and names and brief descriptions of courses and options may be displayed in the content display area D2. In an input area D3, a control method such as a master card to be executed when input of a selection portion R is received may be displayed.

Referring to (b) of FIG. 15, the user may perform input of the executing portion P47 to perform the execution step T3. In this process, the control panel P may detect whether water is insufficient in the steam part 200. When the control panel P detects that water is insufficient in the steam part 200 and generates insufficiency information, the insufficiency information may be displayed through the display portion P8.

The insufficiency information may include a guide to charge the internal supply 400 with water or to open the external supply 500.

Referring to (c) of FIG. 15, the insufficiency information displayed in the pop-up form I may disappear when input of the selection portion R is received or may disappear after the reference time elapses.

Needless to say, the pop-up form I may also be set to disappear when it is confirmed that input of the selection portion R is received or water is supplied to the steam part 200.

(a) and (b) FIG. 16 shows another embodiment of displaying the insufficiency information.

The insufficiency information may be displayed as a whole area or as single information II on the display portion P8.

In other words, only the insufficiency information may be displayed as the single information II on the display portion P8 to make the user aware of insufficiency information and induce an action.

For example, when the selected course and option use steam or the steam course is performed, e.g., a steam part C3 is selected from the setting portion P2, the control panel P may detect whether water is insufficient in the steam part 200. When the control panel P detects that water is insufficient in the steam part 200 to generate insufficiency information, the insufficiency information may also be displayed through the display portion P8.

Referring to (a) of FIG. 16, when the insufficiency information is displayed while the steam course is selected or executed, the display p84 may display the insufficiency information as the single information II.

The single information II may also be removed as the reference time elapses.

However, the single information II may be set to disappear when input of the selection portion R or water supply to the steam part 200 is confirmed.

Referring to (b) of FIG. 16, when the user performs input of the selection portion R, the display portion P8 may return to the state before displaying the insufficiency information.

Thus, the user may disregard the single information II and may also execute the course and the option.

Needless to say, when the user takes an action to supply water to the steam part 200, the insufficiency information displayed on the display portion P8 may be removed.

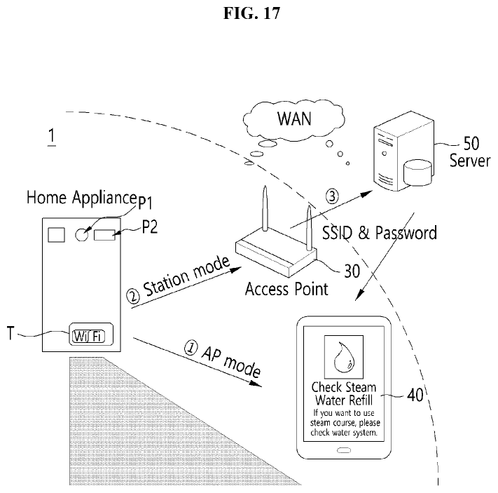

FIG. 17 shows another embodiment of displaying the insufficiency information.

The communication module T of the laundry treating apparatus 10 may communicate with a server 50 provided by a producer through an AP30 such as a router, and may be provided to communicate with an external terminal 40 provided to communicate with a smartphone.

Accordingly, when the control panel P generates insufficiency information, the insufficiency information may be transmitted to the server 50, and may be transmitted to the external terminal 40.

As a result, the server 50 may transmit the insufficiency information to the user through the external terminal 40, and the external terminal 40 may display the insufficiency information as separate information III using a liquid crystal.

Even if the user is positioned at a position spaced apart from the laundry treating apparatus 10, the user may recognize that there is a problem with water supply to the steam part 200 and may take an action to supply water to the steam part 200.

The laundry treating apparatus may perform the course and the option irrespective of whether the insufficiency information is generated.

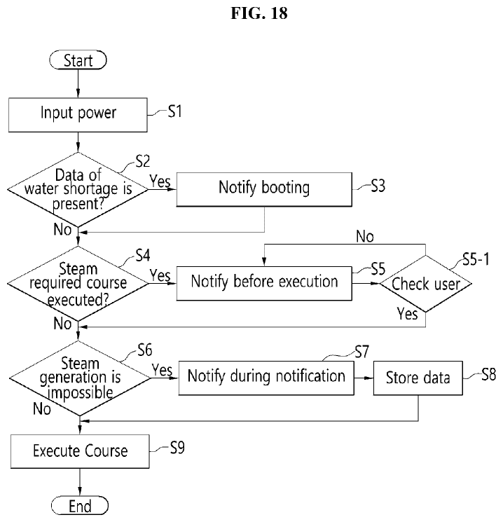

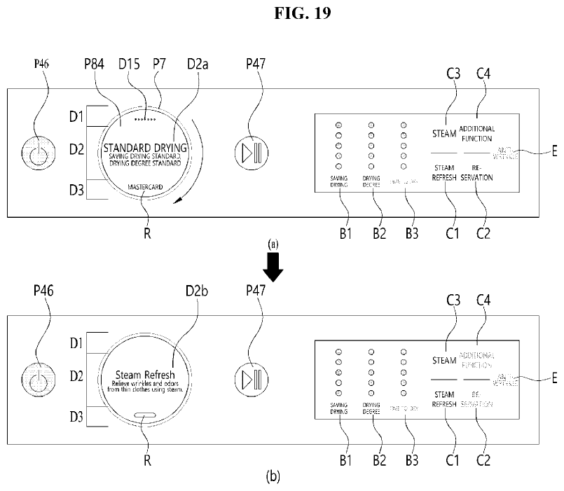

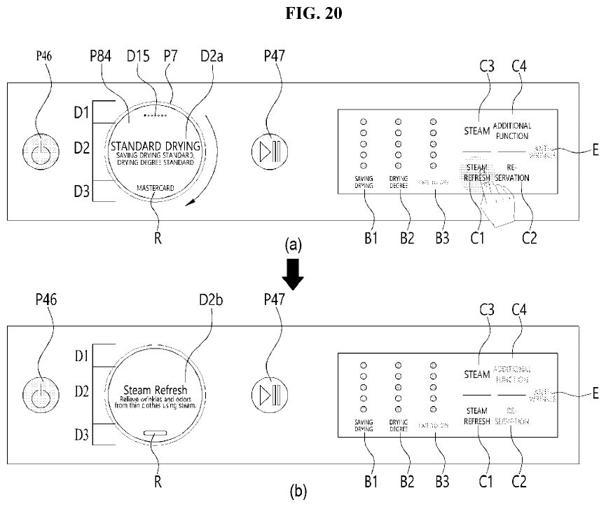

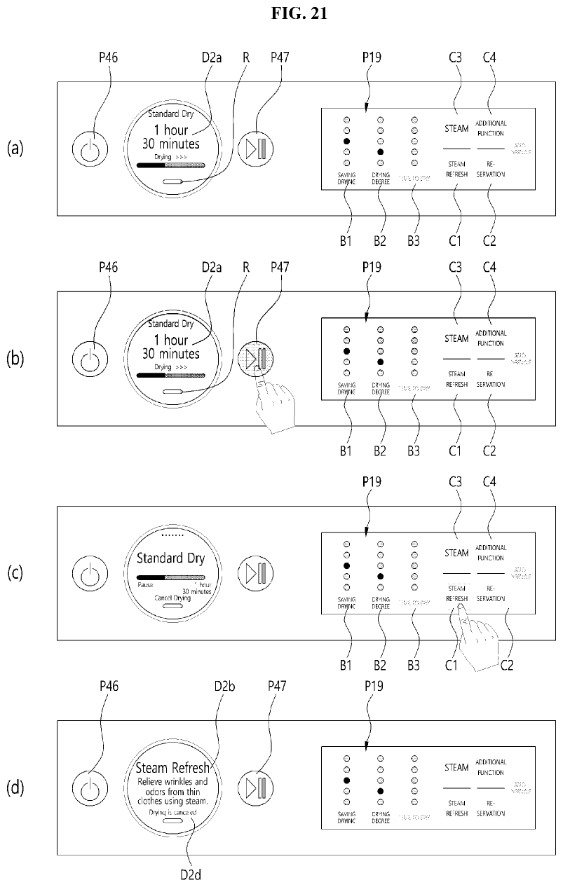

FIG. 18 shows an embodiment of a control method of displaying the insufficiency information.

The laundry treating apparatus according to the present disclosure may perform a power step S1 in which the control panel P detects input of the power switch P46.

When the power step S1 is performed, the controller C may perform a data determination step s2 of determining whether the insufficiency information is stored in the storage H when a previous course or the like is executed.

In the data determination step s2, the controller C may check whether the insufficiency information is the latest information. For example, it can be determined that the insufficiency information is up-to-date if the insufficiency information is present until power is supplied and shut off or is generated during this process.

When the insufficiency information is present in the storage, the controller C may perform a booting notification step s3 of displaying the insufficiency information on the display portion P8 at the booting time point a1.

The display portion P8 may display the insufficiency information as one of the pop-up form I or the single information II.

In the booting notification step s3, the display portion P8 may be configured to remove display of the insufficiency information after the reference time elapses. Thus, the user may select the course and the option without taking an action to supply water to the steam part.

The laundry treating apparatus according to the present disclosure may perform a steam determination step S4 of determining whether to select or execute the steam course of supplying the steam.

The display portion P8 may perform a pre-execution notification step S5 of displaying the insufficiency information at the selection time point a2 when the steam course is selected.

When the steam course is executed, the controller C may detect a water supply condition of the steam part 200 to generate insufficiency information. Thus, the generate insufficiency information may be transmitted to the display portion P8.

When there is insufficiency information that is pre-stored in the storage H, the controller C may display the insufficiency information at the selection time point a2 but not the booting time point. Thus, when the steam course is selected, the controller C may induce the user to supply water to the steam part 200.

When the user select another course other than the steam course, an action of supplying water to the steam part 200 may be induced to be omitted, thereby enhancing convenience of the user and preventing water from being accommodated in the steam part 200 for a long time.

In the pre-execution notification step S5, the display portion P8 may display the insufficiency information as one of the pop-up form I or the single information II.

When the pre-execution notification step S5 is performed, a confirmation step S5-1 in which intention that the user confirms the insufficiency information is reflected may also be performed. In the confirmation step S5-1, the steam course may not be executed after the reference time elapses, but the compulsory intention of the user may be confirmed.

The compulsory intention may be detected through input of the selection portion R or input of the executing portion P47. This is because it is assumed that the user intends to force the steam course when the user performs input of the executing portion P47 to force the steam course or inputs a command for removing the insufficiency information from the display portion P8 through the selection portion R rather than supplying water to the steam part 200 even if the insufficiency information is displayed.

When detecting the compulsory intention in the confirmation step S5-1, the display portion P8 may remove display of insufficiency information and may display an execution state of the steam course.

In other words, because drying of laundry takes priority over execution of the steam course, a drying cycle may be performed first without water in the steam part 200.

Since the user already forces input of the executing portion P47 while recognizing that there is no water in the steam part 200, it may be possible to prevent the drying cycle from being delayed by reflecting the compulsory intention of the user, and the user may be allowed to supply water to the steam part 200 at any desired point.

In order to reliably reflect the compulsory intention, execution or performance of the steam course may be stopped until the compulsory intention is input.

The controller C may perform a steam non-generation detection step S6 in which it is detected that water is not supplied to the steam part 200 during execution of the steam course or the steam option.

When water is not supplied to the steam part 200 in the steam non-generation detection step S6, a notification step during execution S7 in which the controller C generates the insufficiency information and the display portion P8 displays the insufficiency information at the execution time point a3 may be performed.

Thus, the user may recognize the state in which water supply to the steam part 200 is blocked while steam is supplied.

In the execution time point a3, even if the insufficiency information is displayed, the steam course may be continued without stopping. This is because, when the laundry treating apparatus 10 is operated, there is a high possibility that the user is not capable of checking the insufficiency information since the user is likely to be far apart from the laundry treating apparatus 10.

Thus, the display portion P8 may display the insufficiency information only for the reference time and remove display of the insufficiency information again.

However, a storage step S8 of storing the insufficiency information in the storage H may be performed.

As a result, when the insufficiency information is not generated, the course may be executed, and even if the insufficiency information is generated, the course may be executed. As a result, a course execution step S9 may always be completed.

In the laundry treating apparatus according to the present disclosure, when water is not supplied to the steam part 200 until the course is completed, the display portion may display the insufficiency information at the completion time point a4.

When the reference time elapses after the insufficiency information is displayed at the completion time point a4, the display portion P8 may remove display of the insufficiency information. The laundry treating apparatus may shut off power supply.

After the insufficiency information is displayed, the steam part generates steam or water is supplied to the steam part, the controller C may remove display of the insufficiency information displayed on the display portion P8, and may delete insufficiency information stored in the storage H.

However, when the insufficiency information is maintained without being deleted up to the completion time point a4, the controller C may store the insufficiency information C in the storage H and may then shut off power of one or more of the display portion P8 and the driver P28.

FIG. 19 shows a special course and a method of selecting the special course in a laundry treating apparatus according to the present disclosure.

The laundry treating apparatus according to the present disclosure may perform an arbitrary course and option to be executed by the conventional laundry treating apparatus. For example, various courses and options may be performed to remove moisture from wet laundry and make the laundry low in moisture content.

The laundry treating apparatus according to the present disclosure may further include a special course and option by separately applying a new component or control method from the existing course and option.

For example, the laundry treating apparatus according to the present disclosure may supply steam to laundry accommodated inside the drum 2 through the steam part 200. The steam part 200 may generate steam by heating water, and thus may have a temperature higher than the sterilization temperature (e.g., about 60 degrees Celsius) for eradicating bacteria. For example, the steam part 200 may have a temperature corresponding to a boiling point of water.

Thus, when steam is supplied to laundry, bacteria present in the laundry may be eradicated, and odors may be removed from the laundry.

Since steam corresponds to water vapor, when the steam is supplied to the laundry, wrinkles in the laundry may be removed, and foreign substances attached to the laundry may be vaporized or removed together with the water vapor, thereby removing various kinds of odors.

When steam is supplied to the laundry together with hot air generated in the heat exchanger 4, chemicals such as sweat, ammonia, etc. attached to the laundry may be vaporized and removed together with the water vapor.

As a result, the laundry may be refreshed by steam supplied from the steam part 200 and hot air from the heat exchanger 4.

The user may use the laundry treating apparatus to dry moisture contained in the laundry like wet cloth after washing, but may also want to use the laundry treating apparatus to refresh the dry laundry that does not contain moisture.

However, when the dry laundry is input to the drum 2 and the drum 2 is operated, most courses and options of the laundry treating apparatus assume wet laundry, hot dry air may be supplied, and there may be a risk that the dry laundry is damaged by heat.

To prevent this, the laundry treating apparatus according to the present disclosure may be provided by separately developing a special course for performing refresh, e.g., wrinkle removal, odor removal, etc. of the dry laundry.

Hereinafter, the case in which the special course and option are the refresh course will be described, but the case in which the special course is a newly developed course other than the refresh course may not be excluded.

In the refresh course, hot air at a lower temperature than the existing course may be supplied to the drum 2, and since laundry to be refreshed is highly likely to be dry rather than wet, a large amount of steam may be sprayed from the beginning to expose water vapor throughout the laundry.

Thus, the laundry may be prevented from being damaged by hot air from the beginning.

In addition, laundry may be refreshed quickly by rapidly vaporizing the water vapor by hot air after initial steam injection. As a result, it may be possible to significantly save an operating time and input energy of the laundry treating apparatus than when performing a general drying course and option.

The refresh course may be selected by manipulating the manipulation portion P7.

Referring to (a) of FIG. 19, the display portion P8 may display basic information D2a on an arbitrary course and option for drying laundry like a standard course.

Referring to (b) of FIG. 19, the refresh course may appear on the display portion P8 according to a set order by rotating the manipulation portion P7.

Accordingly, the user may rotate the manipulation portion P7 until the refresh course appears on the display portion P8.

The display p84 may display special information D2b related to the refresh course. The special information D2b may be largely displayed in the content display area D2 to allow the user to intuitively recognize this.

The special information D2b may include a phrase, an icon, or a symbol that describe an effect and function of the course as well as a name of the refresh course.

The user may check the special information D2b to know related information as well as presence of the special course and option and may then perform input of the selection portion R or the executing portion P47 to determine to perform the refresh course.

The refresh course may be performed only in the laundry treating apparatus that simultaneously includes the steam part 200 and the heat exchanger 4. Thus, users who are unfamiliar with the laundry treating apparatus 10 according to the present disclosure may not even know existence as well as an effect of the refresh course until the special information D2b is displayed on the display portion P8.

In this state, the refresh course may be selected only by the manipulation portion P7, and thus it may be unlikely that the user uses the refresh course as long as the special information D2b is exposed.

Thus, the laundry treating apparatus according to the present disclosure may include a separate means for displaying and selecting the refresh course.

(a) and (b) of FIG. 20 shows an additional embodiment of selecting a special course.

The laundry treating apparatus may further include a special course part C that is spaced apart from the manipulation portion and configured to receive a command for selecting the special course or option for rotating the drum.

The special course part C may be configured to be separately selected through an auxiliary display p19 of the setting portion P2.

That is, the special course part C may not correspond to an option for changing a condition of an arbitrary course and may be a special course corresponding to the arbitrary course. That is, the special course part C may correspond to an algorithm and a control method throughout the start and end of processing of the laundry treating apparatus.

Nevertheless, the special course part C may be independently provided in an independent area provided separately from the manipulation portion P7 for selecting a course like the auxiliary display p19.

Thus, the user may accurately recognize existence of the special course. When the user does not know a function or effect of the special course, the user may be curious to learn about the special course.

As such, the laundry treating apparatus according to the present disclosure may directly expose the special course to the user, thereby maximizing possibility of using the special course and usability of the special course.

When input of the special course part C is received, the special information D2b may be immediately displayed on the display portion P8, and the special course may be selected.

As a result, when a course is selected through the manipulation portion P7, the display portion may display the basic information D2a of the arbitrary course or option, and when input of the special course part is received, the special information D2b of the special course or the option may be displayed, and thus the selected course may be intuitively displayed to the user.

The basic information D2a may correspond to a name or condition of the course and option.

The user may perform input of the executing portion P47 to immediately execute the special course.

When the special course is provided as a steam refresh course, the special course part C may also be displayed as a name of steam refresh.

The user may be willing to press the special course part C as a test, or to review another course even though the special course is selected.

In this case, when the user rotates the manipulation portion P7, the special information may be removed from the display portion P8 and the basic information D2a on an arbitrary existing course and option may be displayed on the display portion P8.

Thus, the user may easily select both of the special course and the existing course and may also easily change the special course and the existing course.

FIG. 21 shows a control method to be selected and performed when the special course and the existing course are executed.

Referring to (a) of FIG. 21, the existing arbitrary course and option may be currently performed.

Thus, the display portion P8 may display the basic information D2a for executing the arbitrary course and option.

Referring to (b) of FIG. 21, the user may perform input of the executing portion P47 to stop executing the arbitrary course and option.

Before input of the executing portion P47 is received, even if input of the setting portion P2 or the special course part C is received, the input may not be applied to the control panel P.

Thus, even if input of the setting portion P2 or the like is received due to a misinput by a child, a special shock, or vibration, the course and the option may be prevented from being arbitrarily interrupted.

Referring to (c) of FIG. 21, the display portion P8 may display interruption information D2c that the course and option have been interrupted.

Thus, the user may be induced to re-enter the executing portion P47, and the user may be informed that there is an opportunity to enter the setting portion P2, or the like.

In this case, the user may perform input of the special course part C. The special course part C may be performed immediately and continuously while the arbitrary course and option are executed.

When performing a drying cycle, the laundry treating apparatus according to the present disclosure may perform the drying cycle until moisture content of the laundry is reduced to a reference value or less rather than performing the drying cycle for a specific time.

Thus, even if the special course begins while the arbitrary course and option are executed, the special course may also be performed until moisture content of the laundry is reduced to the reference value or less, and thus the special course may not conflict with the arbitrary course and option.

For example, when the special course is the steam refresh course, even if the laundry is still wet during execution of the arbitrary course and option, the laundry treating apparatus according to the present disclosure may perform the special course until the moisture content of the laundry is reduced to the reference value or less while steam is supplied to the laundry and then hot air is supplied thereto.

As a result, the special course and the existing course do not conflict with each other, and thus the special course may be continued in the middle of the existing course.

Referring to (d) of FIG. 21, the display portion P8 may display the special information D2c to inform the user that the special course is selected.

The display portion P8 may display cancellation information D2d that execution of the existing course is cancelled. The cancellation information D2d may be displayed in a pop-up form on the display portion P8, and thus exposure of the special information D2c to the user may not be prevented.

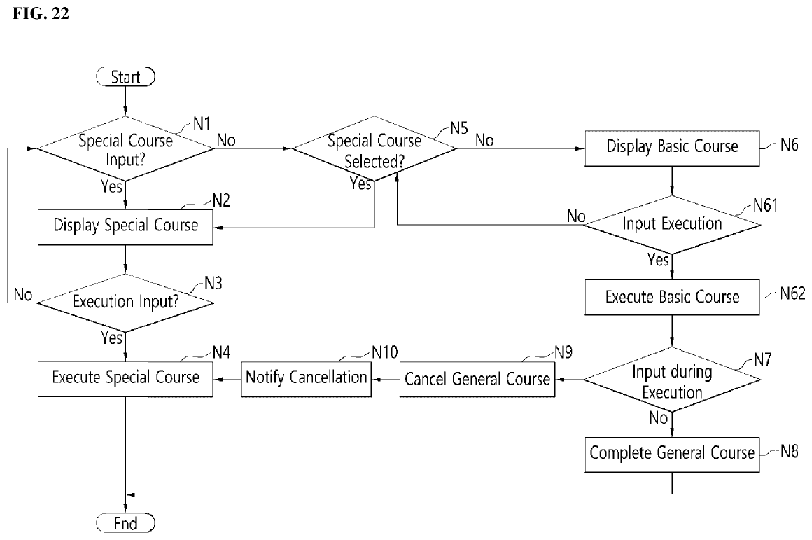

FIG. 22 shows an embodiment of a control method using both of a special course and an existing course by a laundry treating apparatus according to the present disclosure.

When receiving power, the laundry treating apparatus according to the present disclosure may perform an input detection step N1 of detecting whether input of the special course part C is received before the course or the option is executed. When input of the special course part is not received, a selection detection step N5 of detecting whether the special course is selected may be performed through the manipulation portion P7 or the like.

When the special course is selected by the user during the input detection step N1 and the selection detection step N5, the display portion P8 may perform a special display step N2 of displaying the special information D2c of the special course or option.

Through the special display step N2, the user may recognize that a course selected by the user is a special course. The special course or option may be provided separately from an arbitrary course or option selected through the manipulation portion. Thus, in the special display step N2, special information D2d displayed on the display portion P8 may indicate that the special course or option is provided separately from the arbitrary course or option.

For example, in the special information D2d, a specific description phrase of the course may be described differently from the basic course and option.

An execution detection step N3 of detecting whether input of an executing portion N47 is received in the special display step N2 may be performed. When input of the executing portion N47 is received, a special course execution step N4 of executing the special course may be performed.

However, when input of the manipulation portion P7 is detected in the execution detection step N3, the input detection step N1 and the selection detection step N5 may be performed again.

In this case, display of special information may be removed from the display portion P8 by manipulating the manipulation portion P7. The display portion P8 may perform a basic display step N6 of displaying information on an arbitrary existing course or option corresponding to manipulation of the manipulation portion P7. In the basic display step N6, a basic detection step N6-1 of detecting whether input of the executing portion N47 is received may be performed. When input of the executing portion P47 is received in the basic detection step N6-1, a basic execution step N6-2 of executing the existing course may be performed.

When input of the special course part C is received or a special course is selected by the manipulation portion P7 in the basic detection step N6-1, the special display step N2 may be performed again.

While the arbitrary existing course or option is executed in the basic detection step N6-1, a cancellation detection step N9 of stopping and canceling the arbitrary course or option may be performed.

In the cancellation detection step N9, input of at least one of the executing portion P47 and the special course part C may be detected.

For example, when input of the executing portion P47 and the special course part C is received, execution of the existing course and option may be stopped and canceled. During a process of executing the arbitrary course or option, input of the special course part C may be received only when input of the executing portion P47 is first received.

The display portion P8 may perform a cancellation notification step N10 of displaying cancellation information that the arbitrary course or option is cancelled.

In the cancellation notification step N10, the display portion P8 may display the cancellation information for a predetermined time or may display the cancellation information in a pop-up form. When input of the executing portion N47 is received again, the special course execution step N4 of executing the special course may be performed.

That is, when the arbitrary course or option is canceled, the special course or option may be executed.

In the cancellation notification step N10, input of the executing portion P47 is received again, the special course may be performed.

In the cancellation detection step N9, when the arbitrary course and option are not canceled, a completion step N8 performed until the existing course is executed may be performed.

The special course or option may be performed until the laundry is completely dried.

The aforementioned structure, control method, and assembling method of the interface and the laundry treating apparatus are merely examples for describing an example of the interface and the laundry treating apparatus, and the scope of the present application is not limited to the aforementioned embodiments.

Various embodiments described herein may be implemented in a computer-readable medium using, for example, software, hardware, or some combination thereof. For example, the embodiments described herein may be implemented within one or more of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, other electronic units designed to perform the functions described herein, or a selective combination thereof. In some cases, such embodiments are implemented by the controller. For Example, the controller is a hardware-embedded processor executing the appropriate algorithms (e.g., flowcharts) for performing the described functions and thus has sufficient structure. Also, the embodiments such as procedures and functions may be implemented together with separate software modules each of which performs at least one of functions and operations. The software codes can be implemented with a software application written in any suitable programming language. Also, the software codes can be stored in the memory and executed by the controller, thus making the controller a type of special purpose controller specifically configured to carry out the described functions and algorithms. Thus, the components shown in the drawings have sufficient structure to implement the appropriate algorithms for performing the described functions.

For a software implementation, the embodiments such as procedures and functions may be implemented together with separate software modules each of which performs at least one of functions and operations. The software codes can be implemented with a software application written in any suitable programming language. Also, the software codes may be stored in the memory and executed by the controller. Thus, the components shown in the drawings have sufficient structure to implement the appropriate algorithms for performing the described functions.

The present invention encompasses various modifications to each of the examples and embodiments discussed herein. According to the invention, one or more features described above in one embodiment or example can be equally applied to another embodiment or example described above. The features of one or more embodiments or examples described above can be combined into each of the embodiments or examples described above. Any full or partial combination of one or more embodiment or examples of the invention is also part of the invention.

1.-30. (canceled)

31. A laundry treating apparatus comprising:
a cabinet;
a drum rotatably provided inside the cabinet and configured to accommodate laundry therein;
a driver connected to drum and configured to provide power to rotate the drum;
a steam part provided inside the cabinet and configured to generate steam and supply the steam to an inside of the drum;
a water supply connected to the steam part and configured to supply water to the steam part;
a manipulation portion configured to receive a command for selecting a course or option for performing one or more of rotation of the drum or a supply of steam to the inside of the drum;
a controller configured to generate insufficiency information that steam is incapable of being generated by detecting a state in which a supply of water to the steam part is blocked or a supply of water in the steam part is insufficient; and
a display portion coupled to the cabinet and configured to display the insufficiency information,
wherein the display portion is configured to display the insufficiency information at any one of a booting time point at which power is supplied to the display portion, a selection time point of the course or option, an execution time point at which the course or option is executed, and a completion time point at which the course or option is completed, when the insufficiency information is generated.

32. The laundry treating apparatus of claim 31, further comprising a storage configured to store the insufficiency information,
wherein the display portion is further configured to, based on the insufficiency information being present in the storage at the booting time point, display the insufficiency information at the booting time point.

33. The laundry treating apparatus of claim 32, wherein the display portion is further configured to discontinue display of the insufficiency information when a reference time elapses from the booting time point.

34. The laundry treating apparatus of claim 31, wherein the course or option further includes a steam course or a steam option for driving the steam part, and
wherein the display portion is further configured to, when the steam course is selected through the manipulation portion, display the insufficiency information at the selection time point.

35. The laundry treating apparatus of claim 34, further comprising an executing portion configured to receive an execution command for executing the selected course or option,
wherein the display portion is further configured to, based on the execution command being is input, discontinue display of the insufficiency information and display an execution state of the steam course or the steam option.

36. The laundry treating apparatus of claim 34, wherein execution of the steam course or the steam option is completed without change when the insufficiency information is displayed on the display portion.

37. The laundry treating apparatus of claim 36, wherein, based on the insufficiency information being displayed on the display portion, the steam course is stopped, and
wherein, based on display of the insufficiency information being discontinued from the display portion, the steam course is executed again.

38. The laundry treating apparatus of claim 36, wherein the manipulation portion further includes a selection portion configured to receive a command that a user confirms the insufficiency information, and
wherein the display portion is further configured to discontinue display of the insufficiency information upon receiving input of the selection portion.

39. The laundry treating apparatus of claim 38, further comprising an executing portion configured to receive an execution command for executing the selected course or option,
wherein the steam course is stopped until input of the executing portion or the selection portion.

40. The laundry treating apparatus of claim 31, wherein the course or option further includes a steam course or a steam option for driving the steam part, and
wherein the display portion is further configured to, based on the controller generating the insufficiency information during a process in which the steam course or the steam option is executed, display the insufficiency information at the execution time point.

41. The laundry treating apparatus of claim 31, wherein the display portion is further configured to display the insufficiency information at the execution time point, and discontinue display of the insufficiency information when a reference time elapses.

42. The laundry treating apparatus of claim 41, further comprising a storage configured to store the insufficiency information,
wherein, based on the insufficiency information being maintained up to the completion time point, the controller stores the insufficiency information in the storage.

43. The laundry treating apparatus of claim 42, wherein the controller is configured to block power to one or more of the display portion and the driver after storing the insufficiency information in the storage.

44. The laundry treating apparatus of claim 31, wherein the display portion is further configured to, based on the steam part being capable of generating steam or water being supplied to the water supply, discontinue display of the insufficiency information.

45. The laundry treating apparatus of claim 31, further comprising a special course part spaced apart from the manipulation portion and the display portion, the special course part being configured to receive a command for selecting a special course or option for rotating the drum.

46. The laundry treating apparatus of claim 45, wherein the display portion is further configured to, based on receiving input of the manipulation portion, display basic information related to the course or option, and
wherein the display portion is further configured to, based on receiving input of the special course part, display special information related to the special course or option.

47. The laundry treating apparatus of claim 46, wherein the display portion is further configured to, based on receiving input of the manipulation portion in a state in which the special information is displayed, display the basic information again.

48. The laundry treating apparatus of claim 45, wherein the special course or option is additionally provided in the course or option selected through the manipulation portion, and
wherein the display portion is further configured to, based on receiving input of the special course part, display special information that the special course or option is additionally provided in the existing course or option.

49. The laundry treating apparatus of claim 48, wherein the special information includes a name of the special course and option, or a guide about a description or effect of the special course and option.

50. The laundry treating apparatus of claim 45, wherein the special course part is configured to be input in a process in which the course or option is executed, and
wherein, based on input of the special course part being received, execution of the course or option is stopped.

51. The laundry treating apparatus of claim 50, further comprising:
a drying sensor provided in the drum or the cabinet, the drying sensor being configured to detect dryness of the laundry,
wherein the controller is configured to execute the course or option until the laundry is completely dried, and
wherein the controller is configured to also execute the special course or option until the laundry is completely dried.

52. The laundry treating apparatus of claim 50, further comprising an executing portion provided in the cabinet and configured to receive a command for executing or stopping the course or option,
wherein the special course part is configured to be input only based on the executing portion being input in a process in which the course or option is executed.

53. The laundry treating apparatus of claim 52, wherein, based on execution of the course or option being completely cancelled, the special course or option is executed.

54. The laundry treating apparatus of claim 53, wherein the special course or option is executed when the executing portion is input again.

55. The laundry treating apparatus of claim 50, wherein the display portion is further configured to display interruption information that execution of the course or option is stopped or cancelled when the special course part is input to stop or cancel execution of the course or option.

56. The laundry treating apparatus of claim 55, wherein the display portion is further configured to display the interruption information for a predetermined time.

57. The laundry treating apparatus of claim 45, wherein the special course or option includes a supply of steam to the laundry.

58. The laundry treating apparatus of claim 56, further comprising:
a circulation path connected to the drum to circulate air of the drum;
a heat exchanger provided in the circulation path and configured to heat the air; and
a fan provided in the circulation path and configured to move the air,
wherein the special course or option includes a supply of both of hot air and steam to the laundry.

59. The laundry treating apparatus of claim 45, wherein the manipulation portion includes:
a rotary knob rotatably provided at the cabinet, the rotary knob being configured to accommodate the display portion and to receive a command for selecting the course or option; and
a selection portion provided inside the rotary knob and configured to receive a command for determining the selected course or option, and
wherein the special course part is disposed outside the rotary knob.

60. The laundry treating apparatus of claim 59, further comprising a setting portion disposed outside the rotary knob and configured to select the option,
wherein the special course part is disposed inside the setting portion.
